(19) United States
(12) Patent Application Publication
(10) Pub. No.: US 2007/0059783 A1
(43) Pub. Date: Mar. 15, 2007

(54) Method of identifying markers diagnostic of disease and uses therefor in the diagnosis of cancer

(76) Inventors: Nicolle Hannah Packer, Gordon (AU); Niclas Karlsson, Ryde (AU)

Correspondence Address: FENWICK & WEST LLP, SILICON VALLEY CENTER, 801 CALIFORNIA STREET, MOUNTAIN VIEW, CA, 94041, US

(21) Appl. No.: 10/528,745

(22) PCT Filed: Aug. 20, 2003
(86) PCT No.: PCT/AU03/01063

§ 371 Date: Jan. 20, 2006

(30) Foreign Application Priority Data
Aug. 20, 2002 (AU) 2002950878

(51) Int. Cl. G01N 33/574; G01N 33/50; C07K 14/82; C08B 37/00

(52) U.S. Cl. 435/7.23; 436/86; 530/395; 536/53

(57) ABSTRACT

This invention provides methods for the diagnosis of cancer by identifying novel glycans that are diagnostic of cancerous cells or tumors in an human or animal subject, and methods for identifying markers of disease states in humans and/or animals using mass spectrometry on partially purified glycan-containing samples.

### FIELD OF THE INVENTION

This invention relates to the diagnosis of cancer, and, more particularly, to a method for the diagnosis of cancer by identifying novel glycans that are diagnostic of cancerous cells or tumors in any human or animal subject, and optionally, to identify the type of cancer or malignant tumor, by assaying the blood or serum of said subject for an enhanced and/or reduced level of the glycans. The present invention also provides methods for identifying markers of disease states in humans and/or animals using glycosylation profiles of glycans released from biological samples.

### BACKGROUND OF THE INVENTION

General Information

As used herein the term “derived from” shall be taken to indicate that a specified integer may be obtained from a particular source albeit not necessarily directly from that source.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless otherwise stated or an appropriate construction would require otherwise, the integers, steps and features described herein for each embodiment shall be taken to apply mutatis mutandis to every other embodiment.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be-limited in scope by the specific examples described herein. Functionally-equivalent products, compositions and methods are clearly within the scope of the invention, as described herein.

All the references cited in this application are specifically incorporated by reference herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:

1. Sambrook, Fritsch & Maniatis, Molecular Cloning: A Laboratory Manual, Cold Spring Harbor Laboratories, New York, Second Edition (1989), whole of Vols I, II, and III;

2. Immobilized Cells and Enzymes: A Practical Approach (1986) IRL Press, Oxford, whole of text;

3. Handbook of Experimental Immunology, Vols. I-IV (D. M. Weir and C. C. Blackwell, eds., 1986, Blackwell Scientific Publications).

As used herein, “cancer” shall be taken to mean any one or more of a wide range of benign or malignant tumors, including those that are capable of invasive growth and metastase through a human or animal body or a part thereof, such as, for example, via the lymphatic system and/or the blood stream.

As used herein, the term “tumor” includes both benign and malignant tumors or solid growths, notwithstanding that the present invention is particularly directed to the diagnosis or detection of malignant tumors and solid cancers. Typical cancers include but are not limited to carcinomas, lymphomas, or sarcomas, such as, for example, ovarian cancer, colon cancer, breast cancer, pancreatic cancer, lung cancer, prostate cancer, urinary tract cancer, uterine cancer, acute lymphatic leukemia, Hodgkin's disease, small cell carcinoma of the lung, melanoma, neuroblastoma, glioma, and soft tissue sarcoma of humans; and lymphoma (several), melanoma, sarcoma, and adenocarcinoma of animals.

In the context of the present invention as described herein and defined by the claims, the term “marker” shall be taken to mean any molecule that is detectable in a biological sample obtained from a human or animal subject and is indicative of a disorder or disease, or a susceptibility to a disorder or disease in the subject, specifically a glycan or sugar molecule (i.e. a monosaccharide, oligosaccharide or polysaccharide).

The term “cancer marker” shall be, taken to mean a marker that is indicative of cancer in a human or animal subject, specifically a molecule that is produced by or is present on a cancer cell or a normal cell of the subject and whose level is modulated in a sample from a subject having cancer compared to its level in an equivalent sample from a healthy subject. The term “cancer marker” shall also be taken to include (i) a molecule that is expressed specifically by or on a cancer cell or whose expression is enhanced by or on a cancer cell compared to a normal cell ; or (ii) a molecule that is expressed by or on a normal cell but not on a cancer cell, or is shed from a cancer cell, or whose expression is reduced by or on a cancer cells compared to a normal cell.

The term “cancer cell marker” will be understood by those skilled in the art to mean any molecule that is expressed specifically on a cancer cell or whose expression is enhanced on cancer cells compared to normal cells.

The term “cystic fibrosis marker” or “CF marker” shall be taken to mean a marker that is indicative of cystic fibrosis (CF) or an acute pulmonary exacerbation in a subject having CF, specifically a molecule that is present on a high molecular weight glycoprotein that is present in mucus secretions of the subject (eg., as in sputum or saliva) of the subject and whose level is modulated in a sample from a subject having CF compared to its level in an equivalent sample from a healthy subject, or alternatively, is modulated in a CF patient suffering from an acute pulmonary exacerbation relative to its level in a non-CF subject or a CF subject that is not suffering from an acute pulmonary exacerbation. The term “CF marker” shall also be taken to include (i) a molecule that is expressed specifically or at an enhanced level on a high molecular weight mucin such as, for example, MUC2, MUC4, MUC5AC, MUC5B or MUC7 from a CF patient having an acute pulmonary exacerbation, relative to the level of the molecule on a high molecular weight mucin from a non-CF subject or a CF patient that is not suffering from an acute pulmonary exacerbation; or (ii) a molecule that is expressed specifically or at a reduced level on a high molecular weight mucin from a non-CF subject or a CF subject that is not suffering from an acute pulmonary exacerbation relative to the level of the molecule on a high molecular weight mucin from a CF patient that is suffering from an acute pulmonary exacerbation.

The molecular masses of molecules referred to herein are in Daltons, and indicated by the abbreviation “Da”, consistent with accepted nomenclature.

Those skilled in the art will be aware that the term “m/z” refers to the mass-to-charge ratio obtained by dividing the mass of an ion by its charge number.

### BACKGROUND OF THE INVENTION

Markers of Disease

It is widely recognized that simple and rapid diagnostic tests for disease have considerable clinical potential. Not only can such tests be used for the early diagnosis, but they also have general prognostic application in following the course of the disease, including recurrence following treatment.

Accordingly, there is a need for improved methods of identifying markers of particular disease states for use in such diagnostic and prognostic applications.

Cystic Fibrosis Markers

Cystic fibrosis (CF) is one of the most common fatal autosomal recessive disease affecting Caucasian populations. CF has an incidence in neonatals of about 0.05%, indicating a carrier frequency of about 5% of the population. Biological parents of subjects with CF are, by definition, obligatory carriers. Carriers are clinically normal and their detection prior to the birth of an affected child has been precluded by the absence of detectable effects of the gene in single dose.

Methods for detecting CF include DNA sequencing, enzyme immunoassay (Sanguiolo et al., Int. J. Clin. Lab. Res., 25, 142-145, 1995), multiplex DGGE analysis (Costes et al, Hum. Mut. 2, 185-191, 1993), and the use of the polymerase chain reaction (PCR) in conjunction with allele-specific oligonucleotide probes (PCR-ASO). US Patent Application No. 20030008281 (Weston et al.) describes a two tube multiplex ARMS assay for simultaneously detecting 12 of the most prevalent CF mutations in humans, specifically for detecting CFTR gene mutations such as, for example, G542X, W1282X, N1303K, ΔF508, R553X, G551D, R117H, R1162X and R334W (Kazazian et al., Hum Mut. 4, 167-177, 1994), as well as the test distinguishing between CF ΔF508 heterozygotes and homozygotes. The principle of the ARMS test is that the 3′-end of an ARMS amplification primer confers allele-specificity, and an ARMS product is only generated if the primer is complementary to its target at the 3′-end under the appropriate conditions.

CF is a disease of the exocrine glands, affecting most characteristically the pancreas, respiratory system, and sweat glands. The disease usually begins during infancy and the prognosis for an affected child with CF is a median life expectancy currently estimated to be 30 years.

CF is typified by chronic respiratory infection, pancreatic insufficiency, and susceptibility to heat prostration. It is a major cause of death in children. It is estimated that there are between ten million and twelve million carriers for cystic fibrosis in the United States. Each year, between two thousand and three thousand children are born in the United States who are affected by cystic fibrosis. The cost of therapy for cystic fibrosis patients exceeds US$20,000 per year per patient. Of patients diagnosed in early childhood, fewer than fifty percent reach adulthood.

A serious consequence of CF is an exacerbated clinical condition or exacerbated state. As used herein the term “acute clinical exacerbation”, “acute exacerbation”, “clinical exacerbation”, “exacerbation”, or “exacerbated state” in the context of a CF patient shall be understood to mean an exaggeration of a pulmonary symptom of CF.

In most cases, such a clinical exacerbation will be a consequence of a respiratory infection, or increased inflammation. The term “respiratory infection” in this context includes invasion by and/or multiplication and/or colonisation of a pathogenic microorganism in one or more components of the respiratory tract, such as, for example, lung, epiglottis, trachea, bronchi, bronchioles, or alveoli. Commonly, such infections result in the inflammation of the respiratory tract.

CF patients are particularly susceptible to respiratory infections from organisms such as, for example, Staphylococcus aureus, Pseudomonas aeruginosa, Haemophilus influenzae, Aspergillus fumigatus, Burkholderia cepacia complex, Stenotrophomonas maltophila, Alcaligenes (Achromobacter) xylosoxidans, B. gladioli, Ralstonia picketti Influenza A virus and Respiratory Syncytial Virus. The most common causes of respiratory infection are the bacteria S. aureus, P. aeruginosa, and H. influenzae.

For example a chronic respiratory infection, particularly an infection of the lung by P. aeruginosa, accounts for almost 90% of the morbidity and mortality in CF. By age 12, about 60-90% of CF patients are infected with P. aeruginosa.

Progressive loss of pulmonary function over many years due to chronic infection with mucoid P. aeruginosa is common in subjects suffering from CF. Smith et al., Cell. 85, 229-236, 1996, reported defective bacterial killing by fluid obtained from airway epithelial cell cultures of CF patients, and suggested that this phenomenon was due to the inhibition of an unidentified antimicrobial factor resulting from increased levels of sodium chloride in the airway epithelial fluid.

Severe chronic pulmonary disease is also associated with cases of CF wherein CFTR expression on the cell surface is reduced, such as, for example, in patients carrying the ΔF508 mutation. Pier et al. Science. 271, 64-67, 1996 proposed that ingestion and clearance of P. aeruginosa by epithelial cells may protect the lungs against infection, since the specific ingestion and clearance of P. aeruginosa was compromised in a cell line derived from a patient with the ΔF508 mutation.

U.S. Pat. No. 6, 245,735 to Brigham and Womens Hospital disclosed the binding of P. aeruginosa to CFTR via the core portion of the lipopolysaccharide of P. aeruginosa. Also disclosed was a method for up-regulating the CFTR in epithelial mucosa to thereby enhance clearance of P. aeruginosa. Such a method comprises contacting mucosal cells expressing the CFTR with the core portion of the lipopolysaccharide of, P. aeruginosa.

Patients suffering from CF are extremely susceptible to acute clinical exacerbations, often resulting in an increase in inflammation and mucus production, thus increasing the risk of bronchiectasis and eventually respiratory failure.

An acute clinical exacerbation is generally assessed using the protocols described in Williams et al Australian Journal of Physiotherapy, 47, 227-236, 2001; Dakin et al, Pediatr Pulmonol 34, 436-442, 2001; or Rosenfeld et al, J. Pediatr 139 359-365, 2001. In particular, several criteria are assessed, and a patient satisfying four or more of these criteria is considered to have an acute clinical exacerbation. These criteria are Change in sputum production (volume, colour, consistency); new or increased haemoptysis; increased cough; increased dyspnoea (shortness of breath); malaise, fatigue or lethargy; decreased exercise tolerance; fever; anorexia or weight loss; sinus pain/tenderness or change in sinus discharge; FVC or FEV₁ decreased 10% from previous recorded value; radiographic changes indicative of a pulmonary infection; and changes in chest sounds.

Alternatively, an acute clinical exacerbation is also diagnosed using by detecting the concentration of C-reactive protein, determining erythrocyte sedimentation rate, peripheral neutrophil counts and determining serum levels of haptoglobin, as reviewed in Hüner et al, Med Bull Istanbul, 32(1), 1999.

Furthermore, several methods have been suggested for the detection of complications of CF, in particular bacterial infection, more specifically P. aeruginosa infection. Such methods include the monitoring of levels of IgG specific to core lipopolysaccharide (U.S. Pat. No. 5,179,001), IgG specific to P. aeruginosa (Brett et al, J. Clin. Pathol. 39(10) 1124-1129, 1986), and IgA specific to P. aeruginosa (Brett et al, J. Clin. Pathol. 41(10) 1130-1134, 1988). However, these assays require a significant response by the patient's immune system to a P. aeruginosa infection in order to detect these immunoglobulin molecules. Accordingly, such methods are only useful in the monitoring, or prognosis of an established P. aeruginosa infection, rather than being suitable for early detection of infection.

Alternative methods have been suggested for the diagnosis of P. aeruginosa infection in patients suffering from CF, including the detection of P. aeruginosa type-III secretory proteins (Roy-Burman et al, J. Infect. Dis. 183(12), 1767-1774, 2001), the detection of antibodies specific to P. aeruginosa flagellar types a or b in CF patients (Anderson et al, J. Clin. Microbiol. 27(12), 2789-2793, 1989), and the detection of antibodies to sodium alginate exo-polysaccharide of P. aeruginosa in CF patients (Bryan et al, J. Clin. Microbiol. 18(2), 276-282, 1983). Again these assays do not detect early P. aeruginosa infection.

Other pathogens, such as Staphylococcus aureus and non-typable Haemophilus influenzae, are also commonly isolated from the respiratory tract of CF patients.

Whilst there has been significant progress in diagnosing CF, the need still exists for further diagnostic and prognostic assays for complications arising in patients suffering from the disease, in particular rapid and reliable methods for determining whether or not a subject suffering from CF is about to enter an exacerbated condition or state, eg., respiratory infection. Sensitive assays for accurately predicting whether or not a CF patient is entering an exacerbated state, whether this is caused by a microbial infection or not, are highly desirable, as are reliable prognostic indicator for determining whether or not such a subject is responding to treatment for the exacerbated condition.

Mucins constitute a large part of the total protein content of the lung mucosa and whole sputum. They are very high molecular weight glycoproteins, with post-translational oligosaccharide modifications accounting for up to 80% of their total molecular weight. These olhgosaccharides comprise an enormous diversity of structures, which are involved in protein-protein interactions, including mediating leukocyte-pathogen interactions Prakobphol et al. Biochemistry 38, 6817-6825, 1999).

Mucin oligosaccharides are synthesised through the action of a variety of glycosyltransferases, and changes in the activity of these enzymes alter the oligosaccharide structures present (Lamblin, et al. Glycoconjugate J. 18, 661-684, 2001). Fucose, sulfate and sialic acid are typical terminal residues in oligosaccharides, and are therefore important in forming the structures of terminal epitopes which regulate many protein-protein interactions.

There are at least 14 mucin apoproteins encoded by the human genome, designated MUC1, MUC2, MUC3A, MUC3B, MUC4, MUC5B, MUC5AC, MUC6, MUC7, MUC8, MUC11, MUC12, MUC13 and MUC16 (Dekker et al., Trends Biochem Sci. 27, 126-131, 2002). Of these mucins, MUC1, MUC2, MUC4, MUC5B, MUC5AC, MUC7 and MUC8 have been previously detected in human airways, however MUC2, MUC5B and MUC5AC are considered to be major gel-forming mucins in normal and pathological secretions of the airways (Kirkham et al., Biochem. J., 361, 537-546, 2002).

Global alterations in glycosylation of sputum have been associated with CF, but the precise nature of the modified glycosylations, such as, for example, the apoproteins that are modified, has not been elucidated. Nor is known whether or not these global changes are a cause or an effect of CF pathology. In summary, Rhim et al., Glycoconjugate J. 18, 649-659, 2001) suggest that there is enhanced increased fucosylation and decreased sialylation of glycoconjugates generally on epithelial cells, in the absence of a functional CFTR. However, this pattern does not appear in the glycoconjugates of secreted airway mucins (Lamblin et al., Glycoconjugate J. 18, 661-684, 2001)..

Davril et al., Glycobiology 9, 311-321, 1999, described increased sulfation in respiratory and salivary mucins from CF subjects, while increased levels of sialylation and sulfation have also been associated with the severity of infection in CF, as well as in other pulmonary infections. Again, these were global alterations in mucin proteins. Accordingly, the changes reported by Davril et al (1999) may mask specific changes to particular mucin proteins.

Kirkham et al., Biochem. J., 361, 537-546, 2002, also reported an increase in the relative amount of MUC5B relative to MUC5AC in the sputum of CF and asthma patients, and a corresponding increase in a low charge isoform of MUC5B, which was attributed to enhanced submucosal gland secretion of mucins.

Whilst previously identified markers for CF have facilitated efforts to diagnose the disease or an acute pulmonary exacerbation associated therewith, there is a clear need for the identification of additional markers and therapeutic targets. The identification of CF markers that are amenable to the early-stage detection of an acute pulmonary exacerbation is important for more effective management of the genreal state of health of CF patients.

Cancer Markers

Moreover, in spite of numerous advances in medical research, cancer remains a major cause of death worldwide, and there is a need for rapid and simple methods for the early diagnoses of cancer, to facilitate appropriate remedial action by surgical resection, radiotherapy, chemotherapy, or other known treatment method. The availability of good diagnostic methods for cancer is also important to assess patient responses to treatment, or to assess recurrence due to re-growth at the original site or metastasis.

The characterization of cancer markers, such as, for example, oncogene products, growth factors and growth factor receptors, angiogenic factors, proteases, adhesion factors and tumor suppressor gene products, etc, can provide important information concerning the risk, presence, status or future behaviour of cancer in a human or animal subject. Determining the presence or level of expression or activity of one or more cancer markers can assist the differential diagnosis of patients with uncertain clinical abnormalities, for example by distinguishing malignant from benign abnormalities. Furthermore, in patients presenting with established malignancy, cancer markers can be useful to predict the risk of future relapse, or the likelihood of response in a particular patient to a selected therapeutic course. Even more specific information can be obtained by analyzing highly specific cancer markers, or combinations of markers, which may predict responsiveness of a patient to specific drugs or treatment options.

Most known methods for the detection of cancer cells in a subject rely upon the detection of one or more high molecular weight or high molecular mass molecular species in a patient specimen. Immunological assays involve incubating the sample with an antibody molecule, particularly a monoclonal antibody, that binds specifically to a particular cancer cell marker in the sample. Alternatively, genetic tests involve the binding of a nucleic acid probe to nucleic acid in the sample that encodes a proteinaceous cancer cell marker, such as, for example, an oncoprotein. Before the advent of immunological or genetic assays, many cancer cell markers could only be detected or measured using conventional biochemical assay methods, which generally required large test samples and were therefore unsuitable for most clinical applications. In contrast, modern immunoassay and genetic assay techniques can detect and measure cancer cell markers in relatively much smaller samples, including biopsies, or serum.

Notwithstanding the advantages of existing assay techniques for identifying high molecular weight/mass cancer cell markers, such methods require the prior identification of the marker, the prior isolation of an appropriate probe to facilitate subsequent detection of the marker, and a time-consuming binding step in the assay procedure per se. A clear need exists for a rapid throughput method of detecting both high and low molecular weight/mass cancer markers (and cancer cell markers), and that facilitates sample handling and analysis (such as, for example, a process that does not require probe isolation or a binding reaction utilizing such a probe).

Additionally, immunoassays and genetic assays are generally used to determine the presence of a particular cancer cell marker in a sample, possibly because the antigen or nucleic acid detected is tumor-specific. A general method for detecting an enhanced or reduced level of any particular cancer marker is required.

Additionally, notwithstanding that a number of cancer-specific blood tests have been developed which depend upon the detection of tumor-specific antigens in circulation (Catalona et al., New England J Med 324, 1156-1161, 1991; Barrenetxea et al., Oncology 55, 447-449, 1998; Cairns et al., Biochim. Biophys. Acta 1423, C11-C18, 1999), efforts to utilize serum samples generally for cancer marker assays have met with limited success. Such limited success may be because a particular marker is not detectable in serum using immunoassay or genetic screening technology, or because changes in the level of a particular marker cannot be monitored in serum. Clearly, a need exists for a reproducible and reliable process for detecting the presence of cancer markers in serum samples.

Most ovarian cancers are thought to arise from the ovarian surface of epithelium (OSE). Epithelial ovarian cancer is seldom encountered in women less than 35 years of age. Its incidence increases sharply with advancing age and peaks at ages 75 to 80, with the median age being 60 years. The single most important known risk factor is a strong familial history of breast or ovarian cancer. To date, little is known about the structure and function of the OSE cells. It is known that the OSE is highly dynamic tissue that undergoes morphogenic changes, and has proliferative properties sufficient to cover the ovulatory site following ovulation. Morphological and histochemical studies suggest that the OSE has secretory, endocytotic and transport functions which are hormonally-controlled (Blaustein and Lee, Oncol. 8, 34-43, 1979; Nicosia and Johnson, Int. J. Gynecol. Pathol., 3, 249-260, 1983; Papadaki and Beilby, J. Cell Sci. 8, 445-464, 1971; Anderson et al., J. Morphol. 150, 135-164, 1976).

Ovarian cancers are not readily detectable by diagnostic techniques (Siemens et al., J. Cell. Physiol., 134: 347-356, 1988). In fact, the diagnosis of carcinoma of the ovary is generally only possible when the disease has progressed to a late stage of development. A number of proteinaceous ovarian tumor markers were evaluated several years ago, however these were found to be non-specific, and determined to be of low value as markers for primary ovarian cancer (Kudlacek et al., Gyn. Onc. 35, 323-329, 1989; Rustin et al., J. Clin. Onc., 7, 1667-1671, 1989; Sevelda et al., Am. J Obstet. Gynecol., 161, 1213-1216, 1989; Omar et al., Tumor Biol., 10, 316-323, 1989). Several monoclonal antibodies were also shown to react with ovarian tumor associated antigens, however they were not specific for ovarian cancer and merely recognize determinants associated with high molecular weight mucin-like glycoproteins (Kenemans et al., Eur. J. Obstet. Gynecol. Repod. Biol. 29, 207-218, 1989; McDuffy, Ann. Clin. Biochem., 26, 379-387, 1989). More recently, oncogenes associated with ovarian cancers have been identified, including HER-2/neu (c-erbB-2) which is over-expressed in one-third of ovarian cancers (U.S. Ser. No. 6,075,122 by Cheever et al, issued Jun. 13, 2000), the fms oncogene, and abnormalities in the p53 gene, which are seen in about half of ovarian cancers. Oncogene markers are not generally amenable to rapid and simple diagnosis of ovarian cancer, because they may be limited to cancer cell tissues and do not necessarily appear in metastases or in the circulation.

Recently, a GPCR designated as PHOR-1 was identified as having utility in the early detection of prostate cancer (see International Patent Publication No. WO01/25434, Apr. 12, 2001). Expression of PHOR-1 is localized to the prostate gland of healthy individuals, however is up-regulated in prostate tumors and can also be detected in tumors of the kidney, uterus, cervix, stomach and rectum. No other correlation has been recognized between GPCR polypeptide expression and cancer.

Whilst previously identified markers for carcinomas of the lung, prostate, breast, colon, pancreas, and ovary have facilitated efforts to diagnose and treat these serious diseases, there is a clear need for the identification of additional markers and therapeutic targets. The identification of tumor markers that are amenable to the early-stage detection of localized tumors is critical for more effective management of carcinomas of the lung, prostate, breast, colon, pancreas, and ovary.

### SUMMARY OF THE INVENTION

In work leading up to the present invention, the inventors sought to develop improved processes for identifying both high and low molecular weight/mass markers in biological samples from human or animal subjects, and to develop related high throughput diagnostic methods for the detection of disease.

As exemplified herein, the present invention provides improved means and methods for the identification of a marker, based upon changes in the glycosylation profile in certain disease states. This is achieved by partial enrichment/resolution of glycoconjugate molecules from a biological sample of interest, treatment of the released glycoconjugate molecules to release glycans and analysis of the released glycans by mass spectrometry to produce a profile which can then be compared with a control profile. The advantage of this method is that it is not necessary to know the identity of the glycoconjugates from which the glycans are derived nor is it necessary to purify individual molecules since a collective glycan profile can being obtained from a plurality of molecules to provide a representation of glycosylation changes. Furthermore, an important feature of this method is that the glycans are separated from the other constituent parts of the molecule prior to analysis by mass spectrometry and therefore the profile is derived from the released glycans and not the complete glycoconjugates. This provides a less complex profile than if intact glycosylated molecules are subjected to mass spectrometry. The present inventors have demonstrated the general applicability of this technique to the detection of ovarian cancer and the monitoring of cystic fibrosis using plasma samples and sputum samples, respectively.

The inventors found that mass spectrometry (MS) was particularly suited to identifying a range of markers in a bodily fluid from a subject, such as, for example, sputum, saliva, urine, ascites, blood, or serum, specifically glycans or oligosaccharides.

Those skilled in the art will be aware that the adequacy of mass spectrometry, such as, for example, electrospray MS or MALDI-TOF MS, for analysis of any mass of compound, must be determined empirically. This is because the performance of a mass spectrometer is only partially defined by the mass resolution. Other important attributes are mass accuracy, sensitivity, signal-to-noise ratio, and dynamic range. The relative importance of the various factors defining overall performance depends on the type of sample and the purpose of the analysis, but generally several parameters must be specified and simultaneously optimized to obtain satisfactory performance for a particular application. The present inventors have now shown the utility of mass spectrometry to identifying cancer markers in blood or serum fractions, and CF markers in sputum, to aiding the rapid and accurate diagnosis of disease.

Accordingly, one aspect of the present invention provides a method for identifying a marker of an abnormal physiological condition in an individual, which method comprises:
(i) providing a biological sample from the individual;
(ii) subjecting the sample to one or more separation steps to resolve one or more glycoconjugates from other components in the sample;
(iii) treating the one or more glycoconjugates to release glycans;
(iv) analysing the released glycans by mass spectrometry to produce a glycosylation profile; and
(v) analysing the glycosylation profile for changes in a glycan marker which is indicative of the abnormal physiological condition.

Preferably, the high molecular weight glycoconjugate is a high molecular weight glycoprotein, a proteoglycan or a glycolipid.

Preferably step (v) comprises comparing the glycosylation profile produced in step (iv) with a control glycosylation profile.

In a related embodiment, the present invention provides a method for identifying a glycoconjugate whose levels are altered in an individual suffering from an abnormal physiological condition, which method comprises:

(i) providing a biological sample from the individual;

(ii) subjecting the sample to one or more separation steps to resolve one or more glycoconjugates from other components in the sample;

(iii) treating the one or more glycoconjugates to release glycans;

(iv) analysing the released glycans by mass spectrometry to produce a glycosylation profile; and

(v) comparing the profile with a control profile.

(vi) identifying a glycan whose levels are altered in the profile obtained in step (iv) as compared with the control profile; and

(vii) identifying a glycoconjugate present in the biological sample from which the glycan is derived.

In an alternative embodiment, the present invention provides a method for identifying a glycan which is a diagnostic marker for an abnormal physiological condition, which method comprises:

(i) providing a biological sample, from an individual suffering from an abnormal physiological condition;

(ii) subjecting the sample to one or more separation steps to resolve one or more glycoconjugates from other components in the sample;

(iii) treating the one or more glycoconjugates to release glycans;

(iv) analysing the released glycans by mass spectrometry to produce a glycosylation profile; and

(v) identifying a glycan whose levels are altered in the profile obtained step (iv) as compared with a control profile, the identified glycan being the diagnostic marker.

In an alternative embodiment, the present invention provides a method for identifying a glycoconjugate which is a diagnostic marker for an abnormal physiological condition, which method comprises:

(i) providing a biological sample from an individual suffering from an abnormal physiological condition;

(ii) subjecting the sample to one or more separation steps to resolve one or more of glycoconjugates from other components in the sample;

(iii) treating the one or more of glycoconjugates to release glycans;

(iv) analysing the released glycans by mass spectrometry to produce a glycosylation profile;

(v) identifying a glycan whose levels are altered in the profile obtained step (iv) as compared with a control profile; and

(vi) identifying a glycoconjugate present in the biological sample from which the glycan is derived, the identified glycoconjugate being the diagnostic marker.

Preferably, the abnormal physiological condition is a disease state, such as a pathogenic infection, the presence of a malignancy or a respiratory disorder e.g. cystic fibrosis.

In a particularly preferred embodiment, there is provided a method of identifying a cancer marker comprising: (i) obtaining a blood fraction from a human or animal subject having a cancer and from a healthy human or animal; (ii) subjecting the blood fractions to one or more separation steps to resolve one or more glycoconjugates from other components in the sample; (iii) treating the one or more glycoconjugates from each blood fraction to release glycans; (iii) comparing the profile of molecular species obtained from the fractions; and (iv) identifying those molecular species having a modified level, wherein a modified level of said molecular species indicates that the molecular species is a cancer marker.

In an alternative embodiment, this aspect of the invention provides a method for identifying a cancer marker that is indicative of a specific cancer, said method comprising: (i) separating by gel electrophoresis a panel of blood fractions from a panel of human or animal subjects having the cancer to resolve one or more glycoconjugates from other components in the fractions, treating the one or more glycoconjugates from each blood fraction to release glycans thereby forming pools of released glycans, and subjecting each pool of released glycans to mass spectrometry; (ii) separating by gel electrophoresis a blood fraction from a healthy human or animal subject to resolve one or more glycoconjugates from other components in the fraction, treating the one or more glycoconjugates from the blood fraction to release glycans thereby forming a pool of released glycans, and subjecting the pool of released glycans to mass spectrometry; (iii) comparing the profiles of molecular species from each member of said panel of blood fractions at (i) to each other and to the profile of molecular species from the blood fraction at (ii); and (iv) identifying from (iii) those molecular species having a modified level in one member of said panel at (i) when compared to the profile of the blood fraction at (ii), wherein said modified level indicates that the molecular species is a cancer marker that is indicative of a specific cancer.

In an alternative preferred embodiment, there is provided a method of identifying a cystic fibrosis (CF),marker comprising: (i) obtaining sputum or saliva from a CF patient having an acute pulmonary exacerbation and from a non-CF subject or a CF patient that does not have an acute pulmonary exacerbation; (ii) subjecting the sputum or saliva to one or more separation steps to resolve one or more high molecular weight glycoproteins from other components in the sample; (iii) treating the one or more high molecular weight glycoproteins from each subject to release glycans; (iii) comparing the profile of molecular species obtained from the subjects; and (iv) identifying those molecular species having a modified level between a CF patient having an acute pulmonary exacerbation and either a non-CF subject or a CF patient that does not have an acute pulmonary exacerbation, wherein a modified level of said molecular species indicates that the molecular species is a CF marker.

In an alternative embodiment, this aspect of the invention provides a method for identifying a cystic fibrosis (CF) marker, said method comprising: (i) separating by gel electrophoresis a panel of sputum or saliva fractions from a panel of CF patients having an acute pulmonary exacerbation to resolve one or more glycoconjugates from other components in the fractions, treating the one or more glycoconjugates from each subject to release glycans thereby forming pools of released glycans, and subjecting each pool of released glycans to mass spectrometry; (ii) separating by gel electrophoresis a sputum or saliva fraction from a healthy human that does not suffer from CF or is a CF patient that does not suffer from an acute pulmonary exacerbation to resolve one or more glycoconjugates from other components in the fraction, treating the one or more glycoconjugates from the subject to release glycans thereby forming a pool of released glycans, and subjecting the pool of released glycans to mass spectrometry; (iii) comparing the profiles of molecular species from each member of said panel at (i) to each other and to the profile of molecular species from the subject at (ii); and (iv) identifying from (iii) those molecular species having a modified level in one member of said panel at (i) when compared to the profile at (ii), wherein said modified level indicates that the molecular species is a CF marker.

Preferably, a CF marker will be specific for the diagnosis of an acute pulmonary exacerbation in a CF patient.

Preferably, the one or more separation steps are selected from electrophoresis, such as gel electrophoresis, and chromatography. More preferably, the glycoconjugate is subject to one-dimensional gel electrophoresis, in particular one-dimensional SDS-agarose/polyacrylamide gel electrophoresis (1D SDS-AgPAGE)

Typically, the glycans are released from the glycoconjugates by enzymatic and/or chemical means known to the skilled artisan.

In a preferred embodiment, a plurality of glycoconjugates are treated to release glycans. In one embodiment, the total glycoconjugate content of the sample is separated from other components in the sample and treated to release glycans. Preferably, substantially the total glycoprotein and/or proteoglycan content is separated from other components in the sample and treated to release glycans. In an alternative embodiment, not all of the glycoconjugates present in the sample are treated to release glycans.

This method may further comprise identifying and characterising one or more glycans having altered levels in the biological sample from the individual as compared with a control sample.

The method of the invention may be carried out repeatedly over a period of time so as to monitor the development of a particular disorder, e.g. to monitor the progress of a disease, such as an infection, and to assist in determining the optimum clinical regimen for a given patient.

Once markers are identified using the inventive method, the marker can be used to diagnose the disorder and/or monitor the response to treatment of the disorder.

Accordingly, one embodiment of the second aspect of the present invention provides an isolated cancer marker comprises an oligosaccharide comprising a structure selected from the group consisting of:

(i) NeuAc-(Hex-)HexNAc;

(ii) NeuAc-Hex-HexNAc;

(iii) Hex-(Hex-HexNAc-)HexNAc;

(iv) NeuAc-Hex-(NeuAc-)HexNAc;

(v) Hex-(Hex-HexNAc-)HexNAc+NeuAc;

(vi) Hex-HexNAc+NeuAc₃;

(vii) Hex-(Hex-HexNAc-)HexNAc+NeuAc₂;

(viii) Hex₂HexNAc₂(SO₃H)₁;

(ix) Hex₂HexNAc₂NeuAc;

(x) Hex₂HexNAc₂NeuAc(SO₃H);

(xi) DeoxyHex₁Hex₂HexNAc2NeuAc(SO₃H);

(xii) Hex₂HexNAc₂NeuAc₂;

(xiii) DeoxyHex₁Hex₂HexNAc₂NeuAc;

(xiv) Hex₂HexNAc₂NeuAc₂(SO₃H)

or a part thereof.

In an alternative embodiment, there is provided an isolated cancer marker comprising an oligosaccharide that produces as [M-H]⁻ ion in mass spectrometry having a m/z of 1257.2. As will be apparent from the description provided herein, this ionic species comprises multiple sialic acid residues. Preferably it is a disialylated oligosaccharide or trisialylated oligosaccharide. More preferably, this species has the structure Hex-HexNeuAc-NeuAc₃.

In a particularly preferred embodiment, the cancer marker is an ovarian cancer marker.

A third aspect of the present invention provides a method for diagnosing cancer comprising detecting the presence of a cancer marker in a biological sample from a human or animal subject, wherein the cancer marker comprises an oligosaccharide comprising a structure selected from the group consisting of:

(i) NeuAc-(Hex-)HexNAc;

(ii) NeuAc-Hex-HexNAc;

(iii) Hex-(Hex-HexNAc-)HexNAc;

(iv) NeuAc-Hex-(NeuAc-)HexNAc;

(v) Hex-(Hex-HexNAc-)HexNAc+NeuAc;

(vi) Hex-HexNAc+NeuAc₃;

(vii) Hex-(Hex-HexNAc-)HexNAc+NeuAC₂;

(viii) Hex₂HexNAc₂(SO₃H)₁;

(ix) Hex₂HexNAc₂NeuAc;

(x) Hex₂HexNAc₂NeuAc(SO₃H);

(xi) DeoxyHex₁Hex₂HexNAc₂NeuAc(SO₃H);

(xii) Hex₂HexNAc₂NeuAc₂;

(xiii) DeoxyHex₁Hex₂HexNAc₂NeuAc;

(xiv) Hex₂HexNAc₂NeuAc₂(SO₃H)

or a part thereof

In an alternative embodiment, there is provided a method for diagnosing cancer comprising detecting the presence of a cancer marker in a biological sample from a human or animal subject, wherein the cancer marker comprises an oligosaccharide that produces as [M-H]⁻ ion in mass spectrometry having a m/z of 1257.2. Preferably the cancer marker that is detected is a disialylated oligosaccharide or trisialylated oligosaccharide. More preferably, this species has the structure Hex-HexNeuAc-NeuAc₃.

Preferably, the cancer is ovarian cancer.

Preferably, the biological sample comprises blood, or serum.

In a further embodiment of the diagnostic/prognostic methods described herein, the biological sample is obtained previously from the subject. In accordance with such an embodiment, the prognostic or diagnostic method is performed ex vivo.

In yet another embodiment, the subject diagnostic/prognostic methods further comprise processing the sample from the subject to produce a derivative or extract that comprises the carbohydrate analyte (eg., oligosaccharide or glycan).

The present invention further encompasses any suitable assay format for determining modifications to glycosylation profile of a subject's blood or serum in the diagnosis of cancer, specifically ovarian cancer. The diagnostic and prognostic assays described herein are performed using standard assay formats for the detection of monosaccharide or oligosaccharide residues. Immunoassay formats, such as, for example, for the detection of protein or sugars, are particularly preferred. Affinity ligands, such as, for example, lectins that bind specific sugars, can also be used in place of, or alongside, antibodies. Alternatively, or in addition, total carbohydrate content of samples is measured using, for example, Periodic Acid-Schiff's reagent (PAS), whilst the presence of acidic residues is measured using, for example, acetic acid Alcian Blue (aAB) and/or sulfuric acid Alcian Blue (sAB). In this respect, aAB detects sialic acid and sulfate and sAB is specific for sulfate.

High throughput assay formats are also particularly preferred, and immunoassay formats, or detection systems using lectins, or combinations of PAS, aAB and sAB, or mass spectrometry, are particularly useful for this purpose.

The glycan profiling method of the present invention may also be used to identify glycans that can be targeted on a therapeutic basis. For example, a glycan derived from a pathogen glycoconjugate may be used to generate inhibitory molecules that compete with the glycoconjugate for binding to a host molecules.

Accordingly, a further aspect of the present invention provides a method for identifying a candidate therapeutic target, which method comprises:

(i) providing a biological sample from an individual suffering from an abnormal physiological condition;

(ii) subjecting the sample to one or more separation steps to resolve one or more of glycoconjugates from other components in the sample;

(iii) treating the one or more of glycoconjugates to release glycans;

iv) analysing the released glycans by mass spectrometry to produce a glycosylation profile; and

v) identifying a glycan whose levels are altered in the profile obtained in step (iv) as compared with a control profile, the identified glycan being the identified candidate therapeutic target.

In an alternative embodiment, the present invention provides a method for identifying a candidate therapeutic target, which method comprises:

(i) providing a biological sample from an individual suffering from an abnormal physiological condition;

(ii) subjecting the sample to one or more separation steps to resolve one or more glycoconjugates from other components in the sample;

(iii) treating the one or more of glycoconjugates to release glycans;

(iv) analysing the released glycans by mass spectrometry to produce a glycosylation profile; and

(v) identifying a glycan whose levels are altered in the profile obtained in step (iv) as compared with a control profile; and

(vi) identifying a glycoconjugate present in the biological sample from which the glycan is derived, the identified glycoconjugate being the identified candidate therapeutic target.

### BRIEF DESCRIPTION OF THE DRAWINGS

FIG. 1 is a schematic showing the various aspects of the invention.

FIG. 2 shows a glycosylation profile of N-linked oligosaccharides released from a single haptogloblin isomer in plasma separated by 2D gel electrophoresis. The glycosylation profile differs between the normal and diseased states.

FIG. 3 is a photographic representation of one-dimensional (1D) SDS-AgPAGE of high molecular weight glycoproteins from sputum following PAS staining for carbohydrates. Lane I—Cystic Fibrosis subject #1 with acute pulmonary exacerbation; Lane II—Cystic Fibrosis subject #1 recovered after antibiotic/antiinflammatory treatment; Lane II—Normal subject; Lane IV—Cystic Fibrosis subject #2 with acute pulmonary exacerbation; and Lane V—Cystic Fibrosis subject #2 non-responsive to antibiotic/anti-inflammatory treatment. Numbering at the left of the figure indicates molecular weight of mucins.

FIG. 4a is a representation of a mass spectrum showing the glycosylation profile of O-linked oligosaccharides released from a MUC5B-containing mucin fraction of sputum from a healthy non-CF subject. Numbering indicates the m/z ions of different glycans present in the MUC5B-containing mucin fraction.

FIG. 4b is a representation of a mass spectrum showing the glycosylation profile of O-linked oligosaccharides released from a MUC5B-containing mucin fraction of sputum from a CF patient suffering from a clinical exacerbation. Numbering indicates the m/z ions of different glycans present in the MUC5B-containing mucin fraction.

FIG. 4c is a representation of a mass spectrum showing the glycosylation profile of O-linked oligosaccharides released from a MUC5B-containing mucin fraction of sputum from the CF subject indicated in FIG. 4b , following successful treatment for the clinical exacerbation. Numbering indicates the m/z ions of different glycans present in the MUC5B-containing mucin fraction.

FIG. 5 is a representation of a single ion chromatograph of the m/z 1331.2±1.0 ion in FIGS. 4a (left) and 4b (right) following reversed phase HPLC separation of glycans. Differences are observed between the samples. For example, the normal sputum sample shows three distinct isomers of the m/z 1331.2±1.0 ion, whereas the cystic fibrosis sputum sample shows only two isomers.

### DETAILED DESCRIPTION OF THE PREFERRED EMBODIMENTS

Production of Glycan Profiles and Diagnosis/Monitoring of Treatment Efficacy

1. Biological Samples and Reference Samples

Unless otherwise specified, it is preferred that the biological sample that forms the basis of the assays described herein comprises a tissue selected from the group consisting of lung, lymphoid tissue associated with the lung, paranasal sinuses, bronchi, a bronchiole, alveolus, ciliated mucosal epithelia of the respiratory tract, mucosal epithelia of the respiratory tract, squamous epithelial cells of the respiratory tract, a mast cell, a goblet cell, a pneumocyte (type 1 or type 2), broncheoalveolar lavage fluid (BAL), alveolar lining fluid, an intra epithelial dentritic cell, sputum, mucus, saliva, blood, serum, plasma, a PBMC, a neutrophil and a monocyte.

Sputum and saliva are preferred for performance of the diagnostic/prognostic assays of the invention. Sputum can be isolated from lung of a patient using, for example the method described in Gershman, N. H. et al, J Allergy Clin Immunol, 10(4): 322-328, 1999.

In another preferred embodiment a biological sample is plasma that has been isolated from blood collected from a patient using a method well known in the art.

In one embodiment a biological sample is obtained previously from a patient.

2. Sample Resolution

It will be apparent from the preceding description that it is not usual to subject whole sputum, saliva, blood, serum, or whole cells to fractionation by mass spectrometry. Instead, fractions containing the molecular species to be analyzed are loaded into the mass spectrometer. Such fractions can be prepared by standard methods known to those skilled in the art or prepared according to the methods described herein without undue experimentation.

In a preferred embodiment of the method of the first aspect, a biological sample from a subject is subjected to one or more separation steps to resolve one or more of glycoconjugates present in the sample from other components in the sample. However, this step is not essential, and biological samples may be treated to cleave glycans from glycoconjugates without any separation steps.

The term “glycoconjugates” refers to any molecule comprising covalently linked sugar moieties linked to non-sugar moieties. Particular examples include glycoproteins, proteoglycans and glycolipids. Carbohydrates per se, i.e. not linked to non-sugar moieties, are preferably excluded.

Any biological sample comprising glycoconjugates may be used in the method of the present invention. Examples of suitable samples include blood, serum, sputum, tears, urine, saliva, breast milk, and nasal secretions, or fractions thereof The nature of the biological sample chosen will vary depending on the type of condition that is the subject of the diagnostic/prognostic test.

The subject may be any animal, preferably a mammal, and most preferably a human. The subject may also be a non-human animal.

The sample may be treated to remove debris and/or insoluble biological material. For example, samples may be centrifuged and the supernatant used for subsequent testing. Typically, this is carried out as a pre-treatment step prior to subjecting the sample to one or more separation steps to resolve the glycoconjugates,

Separation steps include any technique that involves the separation and/fractionation of the components of the biological sample. Particularly preferred separation methods are electrophoresis or chromatography.

Electrophoresis methods include gel electrophoresis, for example gel electrophoresis using polyacrylamide gels. Electrophoretic separation may be effected on the basis of molecular weight (size) and/or charge. A preferred method of separation on the basis of charge is isoelectric focussing (IEF), preferably using immobilized pH gradients (IPGs). The pH gradients may be narrow (e.g. 1 pH unit or less) or wide (e.g. from pH 2 to pH 12).

It is particularly preferred to use 1D-SDSAgPAGE, or alternatively, a 2D method where IPG-IEF is used for the first dimension and SDS-PAGE is used for the second dimension.

Glycoconjugates may be recovered from the gels using standard techniques, for example the proteins may be transferred to PVDF membranes by electroblotting and portions of the membrane cut out and used for the subsequent analysis steps. Optionally, gels/blots may be stained with reagents that identify sugars, such as Periodic Acid/Schiffs base (PAS).

Depending on the nature of the sample and the electrophoretic technique used, bands/spots identified on the gel will contain more than one glycoconjugate. Where the resolution is such that bands/spots are likely to contain only one molecular species, a number of bands/spots may be picked if it desired to profile a plurality of glycoconjugates.

Chromatographic techniques include separations based on size or charge e.g. gel filtration, size exclusion chromatography, ion-exchange chromatography, and separations based on binding to specific ligands i.e. affinity chromatography. Since the method of the invention involves the resolution of glycoconjugates, an affinity chromatography matrix material that comprises lectins may be particularly convenient since many glycoconjugates will bind to the lectins, leaving other cellular components to be washed through. The purified glycoconjugates may then be recovered with a suitable eluting agent.

Chromatography may be conducted in batch or using columns. Chromatographic fractions may or may not be combined. Optionally, fractions may be concentrated prior to subsequent glycan removal and analysis.

Where the separation technique is designed to resolved glycolipids, a preferred separation technique is thin layer chromatography.

The separation methods need not resolve the glycoconjugates into individual molecular species since the diagnostic method of the invention may advantageously be used to profiling a plurality of glycoconjugates, for example at least 5, 10 or 15 separate species.

3. Glycan Cleavage

The glycans are released from the one or more resolved glycoconjugates using any suitable technique, such as enzymatic and/or chemical means. Specific enzymes that may be used include enzymes that cleave N-linked oligosaccharides (e.g. PNGaseF). Chemical means include the release of O-linked oligosaccharides by reductive beta-elimination.

Released glycans are preferably subjected to a purification step to remove reagents, salts, proteins etc., for example using a graphitised carbon column as described in U.S. Pat. No. 6,376,663.

4. Mass Spectrometry, Profile Generation and Analysis

The glycans are then analysed by mass spectrometry to produce a glycosylation profile. Those skilled in the art will be aware that mass spectrometry is an analytical technique for the accurate determination of molecular weights, the identification of chemical structures, the determination of the composition of mixtures, and qualitative elemental analysis. In operation, a mass spectrometer generates ions of sample molecules under investigation, separates the ions according to their mass-to-charge ratio, and measures the relative abundance of each ion.

Suitable mass spectrometry techniques include time-of-flight (TOF), quadrupole, Fourier transform ion cyclotron resonance, magnetic sector, or quadrupole ion trap.

The general steps in performing a mass-spectrometric analysis are as follows: (i) create gas-phase ions from a sample; (ii) separate the ions in space or time based on their mass-to-charge ratio; and (iii) measure the quantity of ions of each selected mass-to-charge ratio.

Time-of-flight (TOF) mass spectrometers, such as, for example, those described in U.S. Ser. No. 5,045,694 and U.S. Ser. No. 5,160,840, generate ions of sample material under investigation and separate those ions according to their mass-to-charge ratio by measuring the time it takes generated ions to travel to a detector. TOF mass spectrometers are advantageous because they are relatively simple, expensive instruments with virtually unlimited mass-to-charge ratio range. TOF mass spectrometers have potentially higher sensitivity than scanning instruments because they can record all the ions generated from each ionization event TOF mass spectrometers are particularly useful for measuring the mass-to-charge ratio of large organic molecules where conventional magnetic field mass spectrometers lack sensitivity.

The flight time of an ion accelerated by a given electric potential is proportional to its mass-to-charge ratio. Thus the time-of-flight of an ion is a function of its mass-to-charge ratio, and is approximately proportional to the square root of the mass-to-charge ratio. Assuming the presence of only singly charged ions, the lightest group of ions reaches the detector first and are followed by groups of successively heavier mass groups.

TOF mass spectrometers thus provide an extremely accurate estimate of the molecular mass of a molecular species under investigation, and the error, generally no more than 5 Da, is largely a consequence of ions of equal mass and charge not arriving at the detector at exactly the same time. This error occurs primarily because of the initial temporal, spatial, and kinetic energy distributions of generated ions that lead to broadening of the mass spectral peaks, thereby limiting the resolving power of TOF spectrometers. The initial temporal distribution results from the uncertainty in the time of ion formation.

The certainty of time of ion formation is enhanced by utilizing pulsed ionization techniques, such as, for example, plasma desorption and laser desorption, that generate ions during a very short period of time and result in the smallest initial spatial distributions, because ions originate from well defined areas on the sample surface and the initial spatial uncertainty of ion formation is negligible.

Pulsed ionization such as plasma desorption (PD) ionization and laser desorption (LD) ionization generate ions with minimal uncertainty in space and time, but relatively broad initial energy distributions. Because long pulse lengths can seriously limit mass resolution, conventional LD typically employs sufficiently short pulses (frequently less than 10 nanoseconds) to minimize temporal uncertainty.

The performance of LD is enhanced by the addition of a small organic matrix molecule to the sample material, that is highly absorbing, at the wavelength of the laser (i. e. Matrix-assisted laser desorption/ionization, hereinafter “MALDI”). The matrix facilitates desorption and ionization of the sample. MALDI is particularly advantageous in biological applications since it facilitates desorption and ionization of large biomolecules in excess of 100,000 Da molecular mass without their fractionation. A preferred matrix for performing the instant invention comprises 2 (4-hydroxyphenylazo)benzoic acid (HABA), also known as 4-hydroxybenzene-2-carboxylic acid, or dihydroxybenzoic acid (DHB).

In MALDI, samples are usually deposited on a smooth metal surface and desorbed into the gas phase as the result of a pulsed laser beam impinging on the surface of the sample. Thus, ions are produced in a short time interval, corresponding approximately to the duration of the laser pulse, and in a very small spatial region corresponding to that portion of the solid matrix and sample which absorbs sufficient energy from the laser to be vaporized. MALDI provides a near ideal source of ions for time-of-flight (TOF) mass spectrometry, particularly where the initial ion velocities are small. Considerable improvements in mass resolution are obtained using pulsed ion extraction in a MALDI ion source. Ion reflectors (also called ion mirrors and reflectrons, consisting of one or more homogeneous, retarding, electrostatic fields) are also known to compensate for the effects of the initial kinetic energy distribution of the analyte ions, particularly when positioned at the end of the free-flight region. Additional improvements to MALDI are known in the art with respect to the production of ions from surfaces, by improving resolution, increasing mass accuracy, increasing signal intensity, and reducing background noise, such as, for example, those improvements described in U.S. Ser. No. 6,057,543.

The present invention encompasses the use of all modified MALDI-TOF MS systems to determine a marker and/or to aid the diagnosis of disease.

Electrospray MS, or electrospray ionization MS, is used to produce gas-phase ions from a liquid sample matrix, to permit introduction of the sample into a mass spectrometer. Electrospray MS is therefor useful for providing an interface between a liquid chromatograph and a mass spectrometer. In electrospray MS, a liquid analyte is pumped through a capillary tube (hereinafter “needle”), and a potential difference (e. g. three to four thousand Volts) is established between the tip of the needle and an opposing wall, capillary entrance, or similar structure. The stream of liquid issuing from the needle tip is diffused into highly-charged droplets by the electric field, forming the electrospray. An inert drying gas, such as, for example, dry nitrogen gas, may also be introduced through a surrounding capillary to enhance nebulization of the fluid stream. The electrospray droplets are transported in an electric field and injected into the mass spectrometer, which is maintained at a high vacuum. Through the combined effects of a drying gas and vacuum, the carrier liquid in the droplets evaporates gradually, giving rise to smaller, increasingly unstable droplets from which surface ions are liberated into the vacuum for analysis. The desolated ions pass through sample cone and skimmer lenses, and after focusing by a RF lens, into the high vacuum region of the mass-spectrometer, where they are separated according to mass and detected by an appropriate detector (e. g., a photo-multiplier tube). Preferred liquid flow rates of 20-30 microliters/min are used, depending on the solvent composition.

Higher liquid flow rates may result in unstable and inefficient ionization of the dissolved sample, in which case a pneumatically-assisted electrospray needle may be used.

Those skilled in the art will also be aware that it is necessary to prepare the fraction undergoing analysis, for introduction into the MS environment. Preferably, the sample is at least desalted essentially as described in Example 1. More preferably, the sample is fractionated prior to analysis using at least one standard chromatographic separation or purification step. In cases where MALDI-TOF MS is employed, the sample will be mixed with a suitable matrix and dried, whereas in the case of electrospray MS, the sample will be injected directly as a liquid sample in an appropriate carrier solution.

The mass spectrometry profile provides information such as the molecular weight, abundance and/or structure of one or more released oligosaccharides. Any one or more of these characteristics, which differs between the normal and abnormal states, may be used a marker which is indicative of an abnormal conditions. Typically a plurality of differences are used as the basis of a diagnosis, for improved accuracy.

Once the glycosylation profile has been produced, the profile is analysed to identify the presence of a marker which differs from the normal state. Typically, this step of the method involves comparing the glycosylation profile produced from the biological sample of the subject with a control glycosylation profile. The control profile may be generated at the same time as the sample profile or may be a control profile generated previously. Where a profile has been generated previously and a plurality of marker glycan species identified, it may only be necessary to analyse the sample profile for the presence or absence of those particular marker species.

For example, the control may be a healthy subject. As used herein, the term “healthy subject” shall be taken to mean a subject that has not exhibited any symptoms associated with disease when the sample (eg., blood fraction, sputum or saliva, etc) was taken, or is in remission from the symptoms associated with disease at that time, or has not exhibited any recurrence of a previously-diagnosed disease at that time.

Accordingly, the “healthy subject” need not be distinct from the subject suspected of having the disease. For example, a particular individual, such as, for example an individual at risk of developing disease, may provide samples at different times, in which case an early sample taken prior to any symptom development may be used as a control sample against a later sample being tested. Alternatively, a sample taken from a subject in remission, or following treatment, may be used as a control sample against a sample from the same subject taken earlier or later, such as, for example, to monitor the progress of the disease.

By “control sample” is meant a sample having a known composition or content of a particular integer against which a comparison to a test sample is made. The only requirement for the source of a control sample is that it does not contain a level of the marker being detected consistent with disease.

By “comparing the profile of molecular species” is meant that the molecular mass profile of the fraction from the disease sample is compared or aligned to the molecular mass profile of the fraction from the disease sample and the differences noted. Those skilled in the art will be aware that conditions for mass spectrometry of a sample can be manipulated to ensure that the peak height of a particular molecular species, or the area of a particular peak, is proportional to the abundance of that molecular species in the sample. Accordingly, it is not strictly necessary to conduct a further assay of a collected peak sample to determine the abundance of the molecular species therein, because the molecular mass spectra of two samples may be overlaid to determine the differences in peak heights.

Notwithstanding that this may be the case, the present invention clearly includes the step of determining the abundance of any candidate molecular species identified in either the fraction from the subject having disease or the fraction from the healthy subject, and/or the relative abundance of a molecular species in said fractions. This includes determining the abundance or relative abundance of that molecular species in the blood or serum from which the fraction is derived. Standard assays for determining the level of glycan or other oligosaccharide in a sample may be employed, such as, for example, an immunochemical analysis of the peak fraction.

Preferably, the method according to this aspect of the invention includes the further characterization of the disease marker, in particular according to its molecular mass.

The molecular mass (Da) of the disease marker is readily determined by mass spectrometry against standard compounds of known molecular mass, with a maximum error in the estimated molecular mass of 1.0 Da, more preferably 0.5 Da, even more preferably 0.25 Da, still more preferably 0.1 Da, and even still more preferably 0.01 Dalton.

The disease marker may also be further characterized structurally, such as, for example, by fragmentation studies using fragmentation mass spectrometry, such as tandem mass spectrometry (MS/MS) or multiple stage mass spectrometry (MSⁿ), or enzymatic digestion of glycosyl or lipid moities, amongst other techniques known to those skilled in the art.

Preferably, the control glycosylation profile is produced from released oligosaccharides obtained from a biological sample of a subject known to be free of the disorder.

A glycan marker is a characteristic, in terms of molecular weight, abundance or structure. It is apparent that a plurality of glycan markers may be identified for any particular disease.

Glycans and Glycoconjugates Associated with Disorders

The methods of the invention may be used to identify glycans and their associated glycoconjugates whose levels or mass are altered in subjects with a particular disorder.

The techniques for identifying glycan markers differ from the first aspect of the invention only in that prior to the analysis, the markers have not yet been identified and therefore it is essential to include appropriate control samples so that a comparison can be conducted for any molecular species that vary between the sample being tested and the controls.

Typically, a number of sample profiles are compared with a number of control profiles to identify molecular species whose abundance varies between the control profiles and the sample profiles.

Once one or more markers have been identified, it may be desirable to characterise the structure of the glycan using standard techniques, such as MS/MS. Furthermore, the glycoconjugate from which the glycan is derived may also be identified. This is conveniently achieved in the case of profiles produced from glycoconjugates resolved by 2D gel electrophoresis by reference to the spots on the gel that were used to generate the profile. For example, a spot on the gel that gave rise to a particular glycan marker may be analysed by mass spectrometry, following protease digestion, with or without a glycan cleavage step, to identify the protein part of the glycoconjugate.

As discussed above, the glycoconjugates from which the profiled glycans are derived include glycoproteins (for example mucins), proteoglycans, and glycolipids (including glycans). One or more of the glycans may comprise N-acetyl glucosamine, N-acetyl galactosamine, mannose, glucose, galactose, xylose, sialic acid, sulphate, phosphate and/or fucose residues. Preferably, one or more of the glycans comprises sialic acid, sulphate, phosphate and/or fucose residues.

In one embodiment, the glycoconjugates are endogenous to the subject, meaning that they are synthesised by the subject's cells and are not merely present due to infection or foreign bodies. Typically, the glycoconjugates are naturally present on the surface of host cells of the subject and/or are secreted by host cells into bodily fluid. In respiratory disorders such as cystic fibrosis or bronchial infections, the endogenous glycans to be analysed will typically be those present on cells in the mucosal lining of the lungs, or secreted into the mucus lining the lungs.

Of particular interest in the present invention are the glycan portions of the glycoconjugates which are targeted by pathogens and/or host immune cells, and/or which are bound by proteins, such as receptors, present on the surface of said pathogens and immune cells. Herein, this portion is referred to as the epitope of the glycan. In the case of mucin, the epitopes may include Le^{a}, Le^{y}, Le^{x}, sialyl-Le^{x} and 3′-sulpho-Le^{x}.

The results described herein illustrate the identification and characterisation of specific 30 glycan markers associated with ovarian cancer. These specific markers include the two isomers NeuAcα2-6(Galβ-3)GalNAc (galactosylated Sialyl-Tn antigen)(Table 1) and NeuAcα2-3Galβ1-3GalNAc (Sialyl T-antigen) (Table 1) found together with a disialylated structure NeuAcα2-6(NeuAcα2-3)(Galβ-3)GalNAc (Disialyl T-antigen) (Table 1).

A particularly interesting trisialylated oligosaccharide structure was found. It was detected with an [M-H]⁻-ion at m/z 1257.2 (Hex₁HexNAc₁NeuAc₃) (Table 1) at a level of 1% in all the samples analysed.

Thus the presence of these markers in profiles obtained by the methods of the present invention may be used to diagnose ovarian cancer in a patient.

Diagnostic and Prognostic Assay Formats

Any suitable assay format can be used to determine the presence of the glycan markers described herein.

Immunoassay formats, such as, for example, for the detection of sugars, are particularly preferred. In this respect, several antibodies are available publicly that bind to sialic acid, sulfate or facose. For example, antibodies, such as, for example, monoclonal antibody F2 that recognizes the SO₃-3Galβ1-3GlcNAc moiety of the sulfo-Lewis^{a} antigen (Veerman et al., Glycobiol. 7, 37-44, 1997), or the monoclonal antibody INES that binds to less-acidic mucins (Rathman et al., J. Biol. Buccale 181, 19-27, 1990), or monoclonal antibody C241 that binds specifically to sial-Lewis^{a} (Nilsson et al., J., Dermatol. Surg. Oncol. 1, 49-51, 1987), may be useful for antibody-based determinations of sulfate and/or sialic acid contents.

Additional antibodies that bind to sialic acid, sulfated oligosaccharides or fucosylated oligosaccharides, are prepared by standard means. In general, this involves the chemical synthesis of specific oligosaccharides, oligosaccharide substructures, or oligosaccharide epitopes, linkage to a hapten, and immunization of an animal for a time and under conditions sufficient to generate antibodies against the oligosaccharide moiety of the hapten conjugate.

Monoclonal antibodies can be prepared against a peak fraction from mass spectrometry comprising the disease marker, in particular a glycan, and then used in standard immunoassay techniques for the subsequent diagnosis of disease.

In performing this embodiment of the invention, mice or other mammals can be pre-treated by injection with low doses of cyclophosphamide (15 mg/Kg animal body weight) to reduce their suppressor cell activity, and then immunized with various doses of liposome preparations containing glycans, at short intervals (i. e. between 3-4 days and one week), essentially as described in U.S. Ser. No. 5,817,513. Immunizations can be performed by subcutaneous, intravenous, or intraperitoneal injection, in accordance with standard procedures. Before and during the immunization period, animal blood serum samples are taken for monitoring antibody titers generated in the animals against the glycans used as antigens by any known immunoassay method detecting an antigen-antibody reaction. In general, about 5-9 accumulative doses of a liposome preparation at short time intervals will facilitate an antibody response to the glycan. Mice with serum antibody titers against glycans receive a new immunization with the liposome preparations, about three days before obtaining antibody producing cells, and then the antibody producing cells, preferably spleen cells, are isolated.

These cells are fused with myeloma cells to produce hybridomas in accordance with standard procedures for preparing monoclonal antibodies. The titres of the monoclonal antibodies produced by the hybridomas are then tested by immunoassay methods. Preferably, an immuno-enzymatic assay is employed, in which hybridoma supernatants bind to a test sample containing the glycan antigen and then antigen-antibody binding is detected using a second enzyme labelled antibody that binds to the monoclonal antibody. Once the desired hybridoma is selected and sub-cloned, such as, for example, by limiting dilution, the resulting monoclonal antibody can be amplified in vitro in an adequate medium, during an appropriate period, followed by the recovery of the desired antibody from the supernatant. The selected medium and the adequate culture time period are known to the skilled person, or easily determined.

Another production method comprises the injection of the hybridoma into an animal, for example, syngeneic mice. Under these conditions, the hybridoma causes the formation of non-solid tumors, which will produce a high concentration of the desired antibody in the blood stream and the peritoneal exudate (ascites) of the host animal.

Standard immunoassays are then used to assay for the presence of the glycan antigen in a fraction obtained from a subject suspected of having disease. By comparison of the test result to a fraction obtained from a healthy subject, an appropriate diagnosis can be made.

Affinity ligands, such as, for example, selectins or lectins that bind specific sugars, can also be used in place of, or alongside, antibodies. Additionally, the specificity of selectins toward carbohydrates has been extensively reviewed (Rosen et al., Curr. Opin. Cell Biol. 6, 663-673, 1994; Varki, J. Clin. Invest. 99, 158-162, 1997). Various independent research groups have disclosed selectins that recognize carbohydrates incorporating either the sialyl-Lexis^{a}, sialyl-Lewis^{x}, sulfated-Lewis^{x} or sulfated Lewis^{a} structures.

Many lectins are available commercially, such as, for example, from Sigma Chemical Company. For example, a Helix pomantia lectin detects O-linked oligosaccharides such as galNAc; a Maackia amerensis lectin binds to sialic acid; Pseudomonas aeruginosa lectin PA-1 binds to galactose; and Ulex europaeeus lectin binds to α-L-fucose.

Alternatively, or in addition, Periodic Acid-Schiff's reagent (PAS) can be used to measure total carbohydrate content of samples, whilst acetic acid Alcian Blue (aAB) is suitable for determining the presence of acidic oligosaccharides such as, for example, sialic acid and sulfate, and sulfuric acid Alcian Blue (sAB) is suitable for determining sulfate content specifically.

For example, samples from a subject being assayed can be transferred in replica to a suitable matrix eg., PVDF membrane, and each sample stained independently with the following stains (i) Direct Blue to determine total protein content of the sample; (ii) PAS to determine total oligosaccharide content of the sample; (iii) aAB to determine sialic acid and sulfate content of the sample; and (iv) sAB to determine sulfate content of the sample. Stained samples are then imaged according to standard procedures, and the intensity of staining determined. Protein content, oligosaccharide content, acidic oligosaccharide content, and sulfate content per microlitre of sample are determined. The ratio of acidic oligosaccharides to total oligosaccharides (ratio=aAB/PAS), the ratio of sulfate to total oligosaccharide (sAB/PAS), and the ratio of sulfate to acidic oligosaccharide (sAB/aAB) are also calculated directly from the staining obtained.

Optionally, the intensity of staining for each of (i) to (iv) is normalized to the intensity of staining of a standard. Accordingly, the sulfate content, sialic acid/sulfate ratio, total protein and total oligosaccharide content can be rapidly determined for any sample, by simple colorimetric means.

High throughput assay formats are also particularly preferred, and immunoassay formats, or detection systems using lectins, or combinations of PAS, aAB and sAB, or mass spectrometry, are particularly useful for this purpose. A number of samples may be taken over a period of the time and profiles generated so as to monitor the development of a particular disorder, to monitor the progress of a disease, such as an infection. The changes in particular glycan markers can be used to monitor the progress of the development of the disorder and assist clinicians in determining the best clinical practice with respect to an individual patient. This monitoring process may be carried out prior to initiation of treatment and/or as described below, during treatment.

In another embodiment, the glycan profile is used to monitor the progress/efficacy of a treatment regimen. For example, our results show that the glycan profile obtained from a cystic fibrosis patient, with a bacterial infection, following antibiotic treatment differed from the profile obtained prior to treatment. Indeed, the glycan profile obtained from the cystic fibrosis patient following antibiotic treatment became more similar to the profile obtained from a normal patient.

Consequently, the profiling method of the present invention can be used in a similar manner to that described above for diagnosis, to monitor therapeutic treatments by repeated sampling and glycan profiling. Samples are typically taken at one or more time points during treatment and the resulting profile compared with a profile obtained from a sample taken before treatment commenced or at an earlier time point. The changes in particular glycan markers can be used to monitor the progress of the treatment and/or to provide clinicians with a prognosis of the likely outcome of a particular treatment regimen.

The method of the present invention is applicable to diseases or other abnormal physiological conditions which are characterised by altered glycosylation patterns compared to the normal state. Such diseases include cancer, pathogenic infection, autoimmune disease or inflammatory disease. The cancer may be a carcinoma, sarcoma or lymphoma, including cancer of the ovaries, breast, lung, prostrate, liver, brain, urinary tract, pancreas, blood cells, bone marrow, and lymph nodes. The inflammatory disease is preferably associated with pathogenic infection such as pulmonary bacterial or viral infection, which may be caused by one or more of Pseudomonas aeruginosa, Hemophilus influenza and Staphylococcus aureus. Preferably, the pathogenic infection and/or inflammatory disease is one which is associated with a respiratory disorder, for example a disorder characterised by excessive mucus production. Excess mucus production may be due to some physiological defect in the patient, such as in the case of cystic fibrosis, or may be caused by infection by pathogens such as viruses or bacteria, as in the case of tuberculosis or bronchial infections. The excess mucus production may render the patient more susceptible to subsequent bacterial infection. Furthermore, primary and/or secondary infections may result in inflammation mediated by the patient's immune system. Most preferably, the disease is cystic fibrosis, tuberculosis, Crohn's disease, ulcerative colitis or bronchial infections including chronical bronchitis.

Identification of Therapeutic Targets

The identification of glycan markers and optionally the glycoconjugates from which they are derived also provides a means for identifying potential therapeutic targets. Thus, once a glycan marker has been identified as described herein, it may be used to identify potential therapeutic agents for the prevention or treatment of the disorder with which it is associated.

The present invention will now be described further with reference to the following examples which are illustrative only and non-limiting.

### EXAMPLE 1

### Comparison of N-Linked Glycosylation Profile of Normal and Ovarian Cancer Plasma Proteins Separated by 2D Gel Electrophoresis

Methods

Human plasma samples were depleted of some well known abundant proteins, and the proteins were separated by 2D gel electrophoresis, and electro-blotted to PVDF membrane. Identical blots were stained with Direct Blue71 (which stains the majority of proteins) and Periodic Acid/Schiffs (PAS) stain (destructive carbohydrate stain). Glycoproteins were identified from the PAS stained membrane and the corresponding Direct Blue71 protein spots were excised from the blot and treated with PNGaseF O-linked oligosaccharides were subsequently released by beta-elimination and analysed by ESI LC/MS.

Sample Preparation and 2D-Electrophoresis

Human plasma samples were prepared for 2D electrophoresis by depletion of fibrinogen, IgG (immuno-affinity using immobilized protein G (Amersham Pharmacia), and albumin.

Depleted plasma, precipitated with ethanol, was solubilised, and reduced and alkylated. Reduction and alkylation gives you gels which contain less horizontal (false) isoforms and less dimers/trimers.

Electroblotting to PVDF Membrane

Human plasma proteins separated by 2D electrophoresis are electroblotted from the miniChip™ or GelChip™ gels to Immobilon—P^{SQ} PVDF membrane (Millipore). Identical blots of the protein were stained with Periodic Acid/Schiffs base for glycoproteins and Direct Blue (DB71), as a general protein stain. Potential glycoproteins were identified by PAS and the corresponding Direct Blue stained spots which were identified used for glycoproteins analysis.

PNGase F Enzymatic Cleavage and Purification of N-Linked Oligosaccharides

PVDF membrane spots were cut from the membrane and placed in separate wells of a 96 well incubation plate. The spots were then covered with 100 μl of 1% (w/w) PVP (polyvinyl pyrrolidone 40,000) in 50% methanol, and the samples shaken for 20 min before being removed and thoroughly washed with water and placed in a clean, dry well. N-linked oligosaccharides were then cleaved from the treated protein by the addition of 5 μl of PNGase F (0.5 Units/μl) and incubated overnight at 37° C. The sample wells were surrounded by wells containing water to prevent evaporation and the plate sealed.

Oligosaccharides were collected after sonicating the samples (in the 96 well plate) for approximately 5 min. The released N-linked oligosaccharides were transferred from the wells into carbon micro columns.

The carbon micro columns were made from 15 mg of graphitised carbon from an Alltech Carboograph Extract-Clean Tube (Sydney, Australia) which is in methanol, into a ZipTip (Millipore). The Carbon Micro Columns were activated with 3×10 μl of 90% (v/v) MeCN/0.5% formic acid pre-wash, followed by 0.5% formic acid. The N-linked oligosaccharides were trapped on the column by sucking up and down the sample several times. The salts were washed of the column using 3×10 μl of 0.5% formic acid and the N-linked oligosaccharides eluted using 2×10 μl of 25% (v/v) MeCN/0.5% formic acid and dried under vacuum. Before analysis with LC-ESI-MS the samples were re-hydrated.

β-Elimination, Chemical Release and Purification of O-Linked Oligosaccharides

Each spot was wetted with approximately 2 μL methanol. A solution of 50 mM KOH and 0.5M NaBH₄ (20 μL) was added and the spots incubated for 16 hours at 50° C. The samples were neutralised by adding 1 μL Glacial Acetic Acid and transferred into a AG50WX8-cation column (H⁺-form) (BioRad) packed into a Millipore ZipTip, washed with methanol and eluted with 2×60 μL of water and dried under vacuum. The remaining borate was removed by addition of 50 μl of 1% acetic acid in methanol, followed by evaporation. This was repeated a total of five times to ensure all the borate was removed. The samples were then re-suspended in 10 μl of H₂O for LC-MS analysis.

LC-ESI-MS of Oligosaccharides

LC-MS was used for the analysis of both the N and O-linked oligosaccharides. The samples were washed onto the home made column (7 μm Hypercarb particles) or Hypercarb column (5 μm Hypercarb particles), both Thermo Hypersil (Keystone Scientific Operations, Runcorn UK) using a Surveyor auto-sampler. An H₂O- acetonitrile gradient (0-25% Acetonitrile in 30 min, followed by a 3 min wash with 90% acetonitrile) containing 10 mM NH₄HCO₃, was used to separate the oligosaccharides. MS was performed in negative ion mode with three scan events: Full scan with mass range 320-2000 m/z, dependent zoom scan, and dependent MS/MS scan after collision induced fragmentation. Collision conditions used were a normalised collision energy of 40%, activation Q of 0.250 and an activation time of 30 ms. Dynamic exclusion of ions

Results

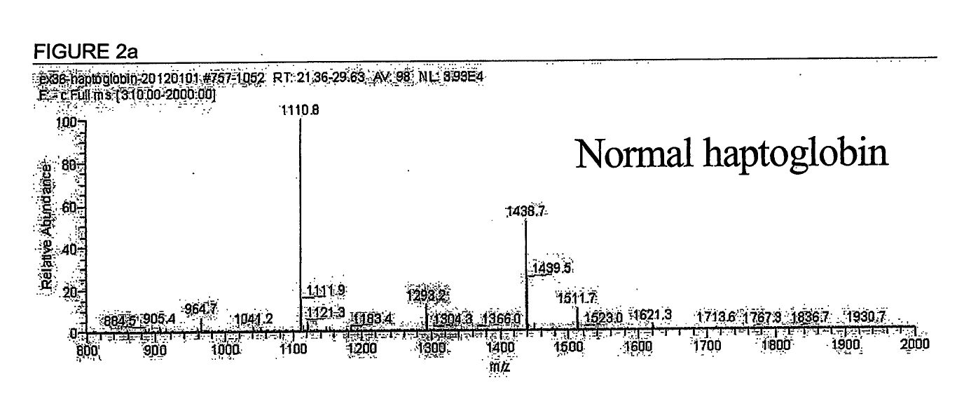

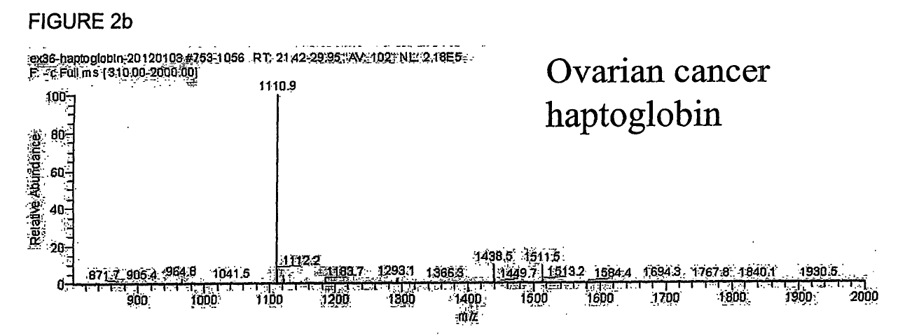

FIG. 2 shows that the N-linked oligosaccharides released from a single haptoglobin isomer separated by 2D electrophoresis results in a different glycosylation profile by mass spectrometric analysis.

### EXAMPLE 2

### Identification of O-Linked Oligosaccharide Marker(s) of Ovarian Cancer

Sample Preparation

Human ascites (500 μL) from a patient with ovarian cancer was reduced (40 mM DTT, 100° C., 30 min) and alkylated (100 mM IAA, RT, 12 h) in 1 mL of sample loading buffer for high molecular weight gels.

The samples (60 μL) were loaded onto 0-6% polyacrylamide, 0.5% agarose composite gels, and run at 100 V for 3.5 h, and blotted onto PVDF membranes. These samples have previously been analysed with western blotting and lectin blotting showing the presence of high molecular weight components (>1 MDa) staining with CA125 antibodies, CA19.9 antibodies and the lectin WGA (sialic acid and GlcNAc). High molecular weight components were also found by staining with PAS and Alcian blue. The Alcian blue stained blot was cut into five bands. The area cut was estimated to include all the components detected by the antibodies and the PAS and Alcian blue stained components from previous experiments. The samples was treated with 0.05 mM KOH/0.5 M NaBH₄ to release attached oligosaccharides, and after desalting and removal of borate the samples were analysed with LC-ESI-MS and LC-ESI-MS/MS.

Results

In Table 1 is a summary of the, detected structures, including the following:

(i) NeuAc-(Hex-)HexNAc;

(ii) NeuAc-Hex-HexNAc;

(iii) Hex-(Hex-HexNAc-)HexNAc;

(iv) NeuAc-Hex-(NeuAc-)HexNAc;

(v) Hex-(Hex-HexNAc-)HexNAc+NeuAc;

(vi) Hex-HexNAc+NeuAc₃;

(vii) Hex-(Hex-HexNAc-)HexNAc+NeuAc₂;

(viii) Hex₂HexNAc₂(SO₃H)₁;

(ix) Hex₂HexNAc₂NeuAc;

(x) Hex₂HexNAc₂NeuAc(SO₃H);

(xi) DeoxyHex₁Hex₂HexNAc₂NeuAc(SO₃H);

(xii) Hex₂HexNAc₂NeuAc₂;

(xiii) DeoxyHex₁Hex₂HexNAc₂NeuAc; and

(xiv) Hex₂HexNAc₂NeuAc₂(SO₃H).

The range is from 3 to 7 oligosaccharide units and the majority of the structures are either sulphated or sialylated, with 1 to 3 of these charge groups attached. Among the smaller ones are the two isomers NeuAcα2-6(Galβ-3)GalNAc (galactosylated Sialyl-Tn antigen)(Table 1) and NeuAcα2-3Galβ1-3GalNAc (Sialyl T-antigen) (Table 1) found together with a disialylated structure NeuAcα2-6(NeuAcα2-3)Galβ-3)GalNAc (Disialyl T-antigen)(Table 1).

In general the glycosylation is similar between the different samples, with a slight tendency towards smaller and less charged oligosaccharides of the high molecular weight components. MS/MS of the oligosaccharides identified some structural features, but was not so successful in assigning sulphated structures. One reason for that is the heterogeneity of these oligosaccharides were for example structures with the composition HSO₃-Hex₂HexNAc₂ were found with up to 10 isomers. Many of the structures have never been described before, especially among the sulphated ones, but also among the other ones where the number of isomers was higher then the number of human structures described in GlycosuiteDB.

The presence of highly charged mucin type molecules could potentially prevent the defence mechanism from coping with cancer, since they could bind to E-selectin and inhibit the selectin-dependent recruitment of leucocytes to the tumor site.

A particularly interesting trisialylated oligosaccharide structure was found. It was detected with an [M-H]⁻-ion at m/z 1257.2 (Hex₁HexNAc₁NeuAc₃) (Table 1) at a level of 1% in all the samples analysed. The structure together with the other data in the table, implies that this is a novel oligosaccharide, possibly a Trisialylated T-antigen, and the data from MS/MS indicates that at least two sialic acids are linked together.

Potentially all the structures found could be used as a marker for ovarian cancer. The galactosylated Sialyl-Tn antigen, the Sialyl T-antigen, and the Disialyl T-antigen are widespread oligosaccharide structures, while many of the others are more rare and could increase the specificity of a diagnostic tool.

TABLE 1
Oligosaccharides detected on human ascites from a patient with ovarian cancer

| Composition | | | | | | Relative Abundance (%) | | | | |
|---|---|---|---|---|---|---|---|---|---|---|
| DeHex | Hex | HexNAc | NeuAc | Sulph | Structure | 1 | 2 | 3 | 4 | 5 |
| | 1 | 1 | 1 | | NeuAc-(Hex-)HexNAc | 0.7 | 1.4 | 1.2 | 5.0 | 1.8 |
| | 1 | 1 | 1 | | NeuAc-Hex-HexNAc | 12.4 | 7.5 | 6.9 | 24.7 | 17.2 |
| | 2 | 2 | | | Hex-(Hex-HexNAc)HexNAc | 1.0 | 0.9 | 0.7 | 2.6 | 1.9 |
| | 2 | 2 | | | | 0.3 | 0.6 | 0.2 | 0.2 | 0.3 |
| | 2 | 2 | | 1 | | 4.9 | 5.4 | 6.2 | 3.3 | 3.1 |
| | 1 | 1 | 2 | | NeuAc-Hex-(NeuAc-)HexNAc | 30.4 | 19.1 | 25.0 | 27.8 | 51.9 |
| | 2 | 2 | 1 | | Hex-(Hex-HexNAc)HexNAc + NeuAc | 1.0 | 0.9 | 0.5 | 0.9 | 0.7 |
| | 2 | 2 | 1 | | Hex-(Hex-HexNAc)HexNAc + NeuAc | 4.2 | 5.5 | 5.2 | 5.4 | 3.8 |
| | 2 | 2 | 1 | | | 0.6 | 0.9 | 1.0 | 0.8 | 0.6 |
| | 2 | 2 | 1 | | | 1.3 | 1.1 | 1.0 | 0.0 | ND |
| | 2 | 2 | 1 | 1 | | 4.4 | 4.2 | 5.3 | 5.3 | 2.1 |
| | 2 | 2 | 1 | 1 | | 5.9 | 5.4 | 6.5 | 5.8 | 3.4 |
| | 2 | 2 | 1 | 1 | | 16.2 | 15.0 | 22.1 | 5.6 | 5.5 |
| | 1 | 1 | 3 | | Hex-HexNAc + NeuAc + NeuAc₂ | 1.2 | 1.2 | 1.1 | 1.4 | 1.1 |
| 1 | 2 | 2 | 1 | 1 | | 0.9 | 0.6 | 0.8 | 0.6 | 0.5 |
| 1 | 2 | 2 | 1 | 1 | | ND | 0.6 | 0.4 | 0.3 | ND |
| 1 | 2 | 2 | 1 | 1 | | 2.3 | 2.5 | 2.5 | 2.5 | 1.3 |
| | 2 | 2 | 2 | | | 1.4 | 0.5 | 0.5 | 0.9 | 0.7 |
| | 2 | 2 | 2 | | Hex-(Hex-HexNAc)HexNAc + 2NeuAc | 2.3 | 1.3 | 1.6 | 1.6 | 1.7 |
| | 2 | 2 | 2 | | Hex-(Hex-HexNAc)HexNAc + 2NeuAc | 8.6 | 22.6 | 5.7 | 4.9 | 2.2 |
| 1 | 2 | 2 | 1 | | | ND | ND | 0.3 | ND | ND |
| 1 | 2 | 2 | 1 | | | ND | 1.1 | 2.4 | 0.4 | 0.2 |
| | 2 | 2 | 2 | 1 | | ND | 0.5 | 1.3 | ND | ND |
| | 2 | 2 | 2 | 1 | | ND | 1.2 | 1.6 | ND | ND |
| | | | | | Sum | 100.0 | 100.0 | 100.0 | 100.0 | 100.0 |
| | | | | | Average length (monosaccharide units) | 4.9 | 5.2 | 5.2 | 4.4 | 4.3 |
| | | | | | Average charge | 1.7 | 1.8 | 1.8 | 1.6 | 1.7 |

### EXAMPLE 3

### Comparison of O-Linked Glycosylation Profile of High Molecular Weight Glycoproteins from a Non-CF Subject, and a CF Subject During and Following an Acute Pulmonary Infection

Materials and Methods

Sputum Collection

Sputum was obtained from healthy and cystic fibrosis patients

Separation of Mucins by SDS-AGPAGE

SDS-AGPAGE gels were made by boiling two solutions with 0.5% agarose and 0.375 M Tris-HCl pH 8.1, one also containing 6% T, 2.5% C (piperazine diacrylamide) and 10% glycerol. The 0-6% gradient polyacrylamide/0.5% agarose gradient gels were cast in the mini-Protean gel casting apparatus (Bio-Rad, Hercules, Calif.) at 50° C. after adding N,N,N′,N′-tetramethylethylenediamine (0.0125%) and ammonium persulphate (0.005%) to each solution. The gels were polymerised for 1 hour at 50° C. and the agarose was then allowed to set at room temperature over-night in a humidified environment. The anode and cathode buffer was 192 mM tris-borate pH 7.6 with 1 mM EDTA and 0.1% SDS.

The sputum was reduced and alkylated in sample loading buffer (Tris-HCl pH 8.1) as described above and sample equivalent to 100 μg Muc2 and 20 μl saliva were loaded onto SDS-AGPAGE gels, and electrophoresed at 100 V for 2-3 hours, until the dye front migrated out of the gel. Proteins were then electroblotted as above, with methanol excluded from the anode buffer. Gels were stained using PAS or Alcian Blue (0.125% Alcian Blue in 25% ethanol and 10% acetic acid for 10 min and destained in 100% methanol for 20 min.

Reductive Alkaline β-Elimination of Oligosaccharides

Oligosaccharides attached to glycoproteins separated by SDS-PAGE or SDS-AGPAGE and blotted to membrane were released by reductive β-elimination. Direct blue or Alcian Blue stained bands were excised from the membrane, wetted with methanol, and incubated at 50° C. for 16 hours in 20 μL 50 mM NaOH and 0.5 M NaBH₄. The resultant solutions were neutralised by the addition of 1 μL glacial acetic acid, before being desalted with 25 μL AG50WX8 cation exchange resin (Bio-Rad) in a zip-tip (Millipore), and dried in a Savant SpeedVac. Borate was removed as its methyl-ester by repeated (5 times) addition and evaporation of 50 μL 1% acetic acid in methanol to each sample. Finally the samples were resuspended in 10 μL MilliQ water for LC-MS analysis.

Mass Spectrometric Identification of Released Oligosaccharides

Desalted oligosaccharides were analysed by LC-MS/MS on a home-made graphitised carbon column 7 μm Hypercarb particles (Thermo-Hypersil, Runcorn UK) in a 250 μm ID column, after introduction using a Surveyor autosampler. A solvent rate through the column of 5 μL/min was provided by a Surveyor LC pump (ThermoFinnigan, San Jose, Calif.) with flow splitting from 100 μL/min. Oligosaccharides were eluted with a H₂O-acetonitrile gradient (0-40% acetonitrile in 30 min, followed by a 3 min wash with 90% acetonitrile) with constant 10. mM NH₄HCO₃. Mass spectrometry was performed on an LCQ Deca (ThermoFinnigan) in negative ion mode, with three scan events: Full scan with mass range 320-2000 m/z, dependent zoom scan of the most intense ions in each scan, and dependent MS/MS scan after collision induced fragmentation. The capillary temperature was 180° C., the capillary voltage was 32.0 V and the electrospray voltage was 2.5 kV. Collision conditions used were a normalised collision energy of 40%, and an activation time of 30 ms. Dynamic exclusion of ions for zoom scan for 30 s was introduced after 3 selections within 30 s. For MS/MS the normalised collision energy was 35% with an activation time of 30 ms.

Results and Discussion

Analysis of Mucin Oligosaccharides.

As compared to smaller glycoproteins, mucins (>200 kDa) are predominantly glycosylated with O-linked oligosaccharides, with up to 80% of the weight. Mucins found on mucosal surfaces are supposed to be important interaction molecules due to their glycosylation. Traditionally, characterisation of oligosaccharides from mucin is carried out after isolation of mucin fractions with isopycnic centrifugation, followed by gel- and anion exchange chromatography (ref). As a final step the oligosaccharides are released and characterised using mass spectrometry, monosaccharide composition analysis, and ¹H-NMR. The approach, taken here is using a highly resolving agarose-polyacrylamide composite gel for isolation of mucin fraction, since high molecular weight of most mucins make them unsuitable for traditional SDS-PAGE. Limited characterisation of released oligosaccharides using high resolving LC-MS, provides sufficient information for identifying glycosylation from well defined mucin subpopulations. Another advantage of the methodology is that a lower amount of mucin will be consumed for the analysis i.e. less than 100 μg of a crude mucin fraction, compared to several milligram of purified mucin for the traditional analysis. The utility of the method described here for glycosylation analysis of gel separated mucins has been validated by comparison with previously reported glycosylation profiles of rat mucins.

The method described here profiles the oligosaccharide structures found on a mucin glycoprotein species from healthy and cystic fibrosis patient sputum.

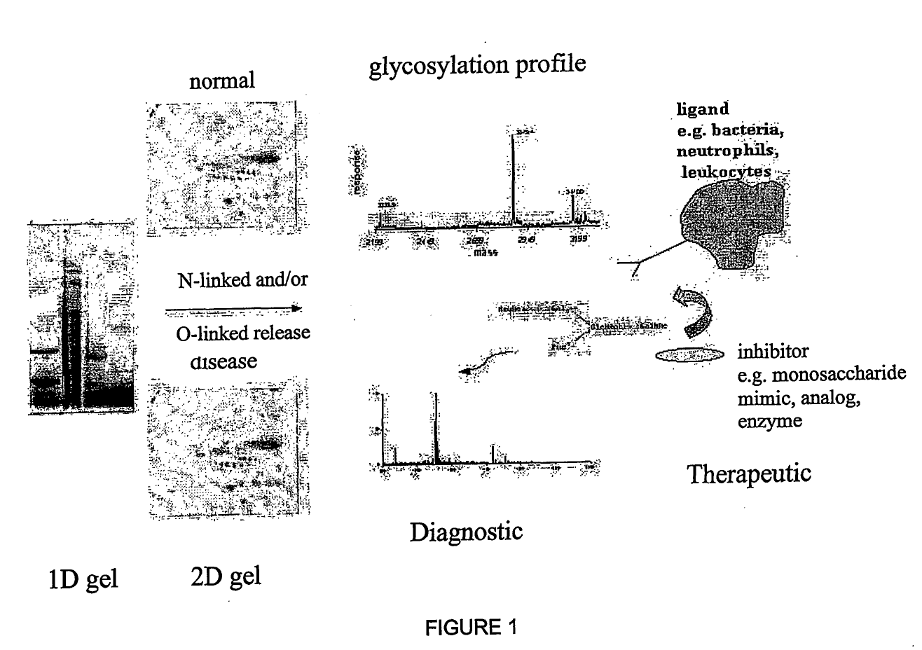

O-linked glycosylation profiling of oligosaccharides released from mucin separated by 1-D AgPAGE gel electrophoresis shows marked differences between CF and normal sputum in both the gel pattern and the glycosylation of the high molecular weight glycoproteins. FIG. 1 shows that acute pulmonary exacerbation of two cystic fibrosis patients (Lanes I and IV) results in the high molecular weight glycoprotein bands of sputum separating at a apparent lower molecular mass on AgPAGE gels than that of non-CF sputum (Lane III). Interestingly, the successful treatment of the pulmonary infection with antibiotics and anti-inflammatories results in the glycoprotein bands appearing in the CF patient sputum sample at the same high molecular weight as those of non-CF sputum (Lane II). In addition, the sputum of a CF patient who did not respond to treatment still maintains the appearance of lower molecular mass glycoproteins (Lane V).

Correspondingly, the total ion mass spectra of the O-linked oligosaccharides released from the high molecular weight glycoproteins was determined (FIGS. 2a-2c). Data presented in FIGS. 2a-2c show differences in the relative amounts of the different oligosaccharide ions present in MUC5B and MUC5AC-containing bands from 1-D AgPAGE profiles of sputa from a non-CF subject compared to a CF patient with an acute pulmonary infection.

The MUC5B-containing fraction from non-CF subjects routinely showed abundant structures having, for example, m/z 628.9±1.0, m/z 701.9±1.0, m/z 710.1±1.0, m/z 782.8±1.0, m/z 811.4±1.0, m/z 878.1±1.0, m/z 884.5±1.0, m/z 957.6±1.0, m/z 965.3±1.0, m/z 1032.3±1.0, m/z 1038.4±1.0, m/z 1120.2±1.0, m/z 1178.2±1.0 and m/z 1266.1±1.0, which were either not present, or present at a relatively low level compared to other structures, in the MUC5B-containing fraction of sputum from the CF subject during a pulmonary exacerbation. The level of the sulfated oligosaccharides having m/z 1032.3±1.0 and m/z 1266.1±1.0 were not reproducibly present at significant levels in sputum of CF subjects during a clinical exacerbation.

Structures having m/z 665.2±1.0, m/z 738.3±1.0, m/z 790.2±1.0, m/z 848.1±1.0, m/z 960.7±1.0, m/z 993.8±1.0, m/z 1040.5±1.0, m/z 1186.4±1.0, m/z 1244.4±1.0, m/z 1322.1±1.0, m/z 1331.3±1.0, m/z 1469.2±1.0, m/z 1477.4±1.0 and m/z 1551.3±1.0 were relatively high in abundance during pulmonary exacerbation in the CF subject (FIG. 2b) compared to the non-CF healthy subject (FIG. 2a). Of these structures, those having m/z 665.2, m/z 738.3±1.0, m/z 960.7±1.0, m/z 993.8±1.0 and m/z 1469.2±1.0, were also not reproducibly detectable at significant levels above background following treatment (FIG. 2c), suggesting that they may be related to the course of infection. Those structures having m/z 993.8±1.0, m/z 1244.4±1.0, and m/z 1477.4±1.0 were amongst the most abundant species in the MUC5B-containing fraction from the CF patient during infection.

Interestingly, the level of m/z 1040.5±1.0 and m/z 1186.4±1.0 and m/z 1331.3±1.0 were high in the CF subject during exacerbation and following treatment, and may reflect an overall CF-related modification to MUC5B, or alternatively, genetic differences between the individuals tested.

Yet other oligosaccharides of MUC5B in the CF-subject following treatment of infection with antibiotics and anti-inflammatories were increased in relative abundance, without reference to the non-CF subject altered (compare FIGS. 2b and 2c

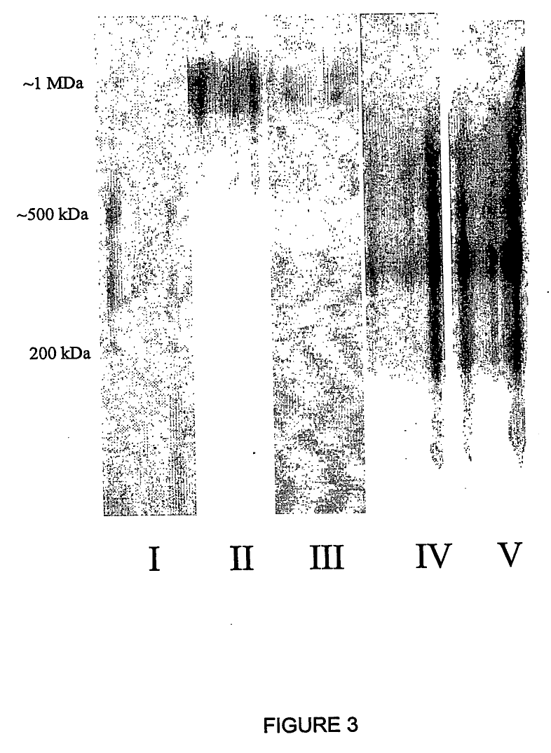

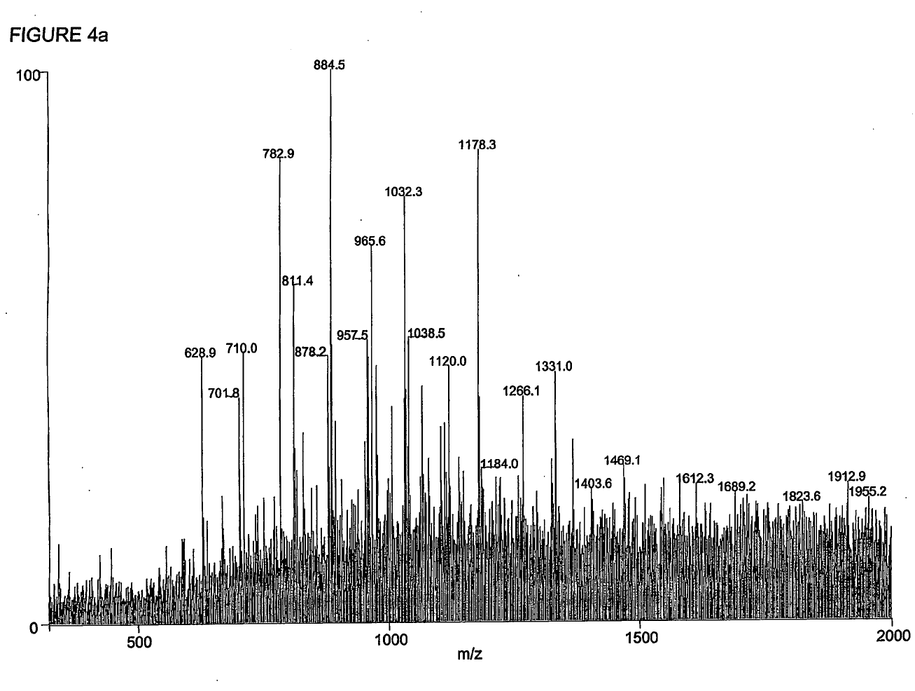

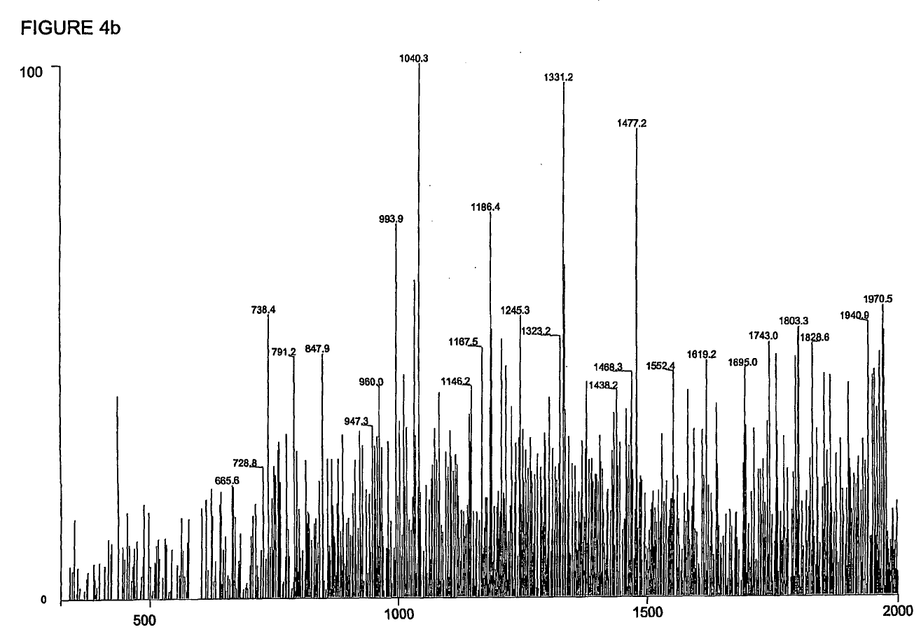

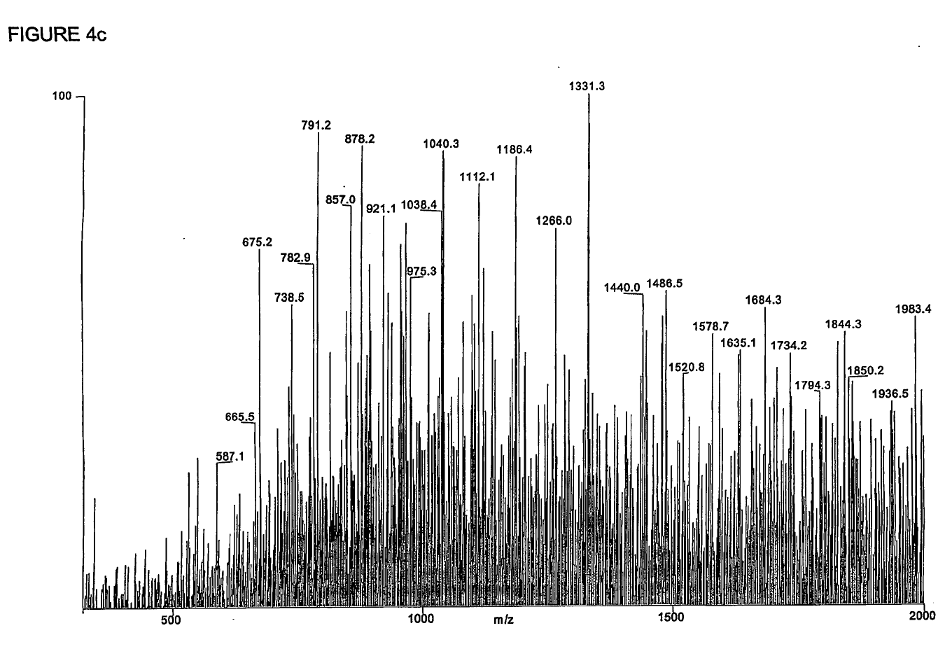

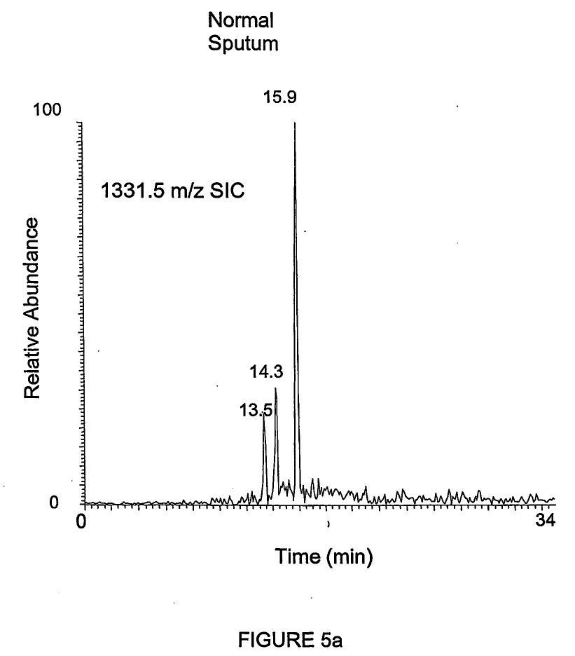

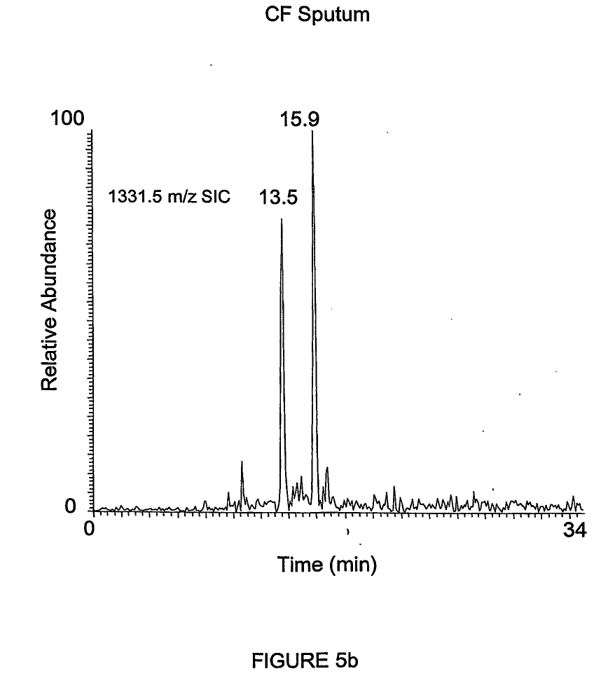

Analysis of single ion chromatographs from the reversed phase HPLC separation of the glycans also shows differences between samples. For example, the m/z 1331.3±1.0 ion the normal sputum sample showed three distinct isomers (FIG. 3a), while only two isomers were detected from the CF sputum sample (FIG. 3b).

Those skilled in the art of mass spectrometry are aware that the but the observered m/z, z, massₙₑᵤₜᵣₐₗ and mass_{predicted} may depend on the analysis technique used, for example, sample preparation and mass spectrometry, and, as a consequence, the reported mass-to-charge ratios reported herein may not arise if different analytical techniques are used to those described. On the other hand, oligosaccharide compositions for each ion compositions would remain the same independent of the analysis technique employed. Accordingly, the inventors determined the oligosaccharide compositional data for each ion identified in the present studies. The compositions included the reducing end, GalNacol, as a HexNac group.

Proceeding on this basis, structures that were routinely enhanced in the MUC5B-containing fraction from non-CF subjects, relative to exacerbated CF subjects, had the following compositions: HexNac₃Hex₂Fuc₁Sulf₁ (m/z 628.9±1.0); HexNac₃Hex₂Fuc₂Sulf₂ (m/z 701.9±1.0); HexNac₃Hex₃Fuc₁Sulf₂ (m/z 710.1±1.0); HexNac₃Hex₂Fuc₂Sulf₂ (m/z 782.8±1.0); HexNac₄Hex₃Fuc₁Sulf₂ (m/z 811.4±1.0); HexNac₂Hex₁NeuAc₁ (m/z 878.1±1.0); HexNac₄Hex₃Fuc₂Sulf₂ (m/z 884.5±1.0); HexNac₄Hex₃Fuc₃Sulf₂ (m/z 957.6±1.0); HexNac₄Hex₄Fuc₂Sulf₂ (m/z 965.3±1.0); HexNac₃Hex₂Sulf₁ (m/z 1032.3±1.0); HexNac₄Hex₄Fuc₃Sulf₂ (m/z 1038.4±1.0); HexNac₂Hex₂NeuAc₁Sulf₁ (m/z 1120.2±1.0); HexNac₃Hex₂Fuc₁Sulf₁(m/z 1178.2±1.0) and HexNac₂Hex₂Fuc₁NeuAc₁Sulf₁ (m/z 1266.1±1.0). The level of the sulfated oligosaccharides having compositions HexNac₃Hex₂Sulf₁ and HexNac₂Hex₂Fuc₁NeuAc₁Sulf₁ were not reporducibly present at significant levels in sputum of CF subjects during a clinical exacerbation.

On the other hand, structures that were routinely and significantly enhanced in abundance during pulmonary exacerbation in CF subjects, relative to non-CF subjects, had the following compositions: HexNac₂Hex₂NeuAc₂ (m/z 665.2±1.0); HexNac₂Hex₂Fuc₁NeuAc₁ (m/z 738.3±1.0); HexNac₃Hex₁ (m/z 790.2±1.0); HexNac₃Hex₃Fuc₂NeuAc₁ (m/z 848.1±1.0), HexNac₃Hex₃Fuc₁NeuAc₂Sulf₁ (m/z 960.7±1.0); HexNac₃Hex₃Fuc₂NeuAc₂ (m/z 993.8±1.0); HexNac₂Hex₂NeuAc₁(m/z 1040.5±1.0), HexNac₂Hex₂Fuc₁NeuAc₁ (m/z 1186.4±1.0); HexNac₃Hex₂Fuc₂ (m/z 1244.4±1.0); HexNac₃Hex₂Fuc₂Sulf₁ (m/z 1322.1±1.0); HexNac₂Hex₂NeuAc₂ (m/z 1331.3±1.0); HexNac₂Hex₂Fuc₁NeuAc₁Sulf₁ (m/z 1469.2±1.0); HexNac₂Hex₂Fuc₁NeuAc₂ (m/z 1477.4±1.0) and m/z HexNac₃Hex₃Fuc₁NeuAc₁ (1551.3±1.0). Of these structures, those the following compositions were also not reproducibly detectable at significant levels above background following treatment: HexNac₂Hex₂NeuAc₂ (m/z 665.2±1.0); HexNac₂Hex₂Fuc₁NeuAc₁ (m/z 738.3±1.0); HexNac₃Hex₃Fuc₁NeuAc₂Sulf₁ (m/z 960.7±1.0); HexNac₃Hex₃Fuc₂NeuAc₂ (m/z 993.8±1.0); HexNac₃Hex₂Fuc₂Sulf₁ (m/z 1322.1±1.0); and HexNac₂Hex₂Fuc₁NeuAc₁Sulf₁ (m/z 1469.2±1.0), were also not reproducibly detectable at significant levels above background following treatment, suggesting that they may be related to the course of infection. Those structures having the following compositions were amongst the most abundant species in the MUC5B-containing fraction from CF patients during infection: HexNac₃Hex₃Fuc₂NeuAC₂ (m/z 993.8±1.0); HexNac₂Hex₂NeuAc₁ (m/z 1040.5±1.0), and HexNac₂Hex₂Fuc₁NeuAc₂ (m/z 1477.4±1.0).

The following compositions were present in CF subjects during exacerbation and following treatment: HexNac₂Hex₂NeuAc₁ (m/z 1040.5±1.0) and HexNac₂Hex₂Fuc₁NeuAc₁ (m/z 1186.4±1.0) and HexNac₂Hex₂NeuAc₂ (m/z 1331.3±1.0).

These representations of the glycosylation profiles of specific separated proteins, or protein mixtures, can be used separately or in combination to compare different sample types or disease states.

Diagnosis or prognosis of pulmonary infections/inflammations based on the compositional data provided compare the measured masses/compositions for a test sample with the masses/compositions listed for each of the groups of subjects. Alternatively, or in addition, a weighted average oligosaccharide composition is calculated based upon the data provided.

1-25. (canceled)

26. A method for diagnosing cancer comprising detecting the presence of a cancer marker in a biological sample from a human or animal subject, wherein the cancer marker comprises an oligosaccharide that comprises two or more linked sialic acid residues.

27. The method of claim 26 wherein the cancer marker that is detected is a disialylated oligosaccharide or trisialylated oligosaccharide.

28. The method of claim 26 wherein the cancer marker that is detected has the structure Hex-HexNeuAc-NeuAc₃.

29. The method of claim 26 wherein the cancer is ovarian cancer.

30. The method of claim 26 wherein the biological sample comprises blood or serum.

31-33. (canceled)

34. The method of claim 26 wherein the cancer marker is detected by a process comprising staining the cancer marker with a dye that binds to the cancer marker.

35. A method for identifying a candidate therapeutic target, which method comprises:
(i) providing a biological sample from an individual suffering from an abnormal physiological condition;
(ii) subjecting the sample to one or more separation steps to resolve one or more of glycoconjugates from other components in the sample;
(iii) treating the one or more of glycoconjugates to release glycans;
(iv) analysing the released glycans by mass spectrometry to produce a glycosylation profile; and
(v) identifying a glycan whose levels are altered in the profile obtained in step (iv) as compared with a control profile, the identified glycan being the identified candidate therapeutic target.

36. The method of claim 35 further comprising identifying a glycoconjugate present in the biological sample from which the glycan is derived, the identified glycoconjugate being the identified candidate therapeutic target.

37. The method of claim 26 wherein the cancer marker comprises an oligosaccharide comprising a structure selected from the group consisting of:
(i) NeuAc-(Hex-)HexNAc;
(ii) NeuAc-Hex-HexNAc;
(iii) Hex-(Hex-HexNAc-)HexNAc;
(iv) NeuAc-Hex-(NeuAc-)HexNAc;
(v) Hex-(Hex-HexNAc-)HexNAc+NeuAc;
(vi) Hex-HexNAc+NeuAc₃;
(vii) Hex-(Hex-HexNAc-)HexNAc+NeuAc₂;
(viii) Hex₂HexNAc₂(SO₃H)₁;
(ix) Hex₂HexNAc₂NeuAc;
(x) Hex₂HexNAc₂NeuAc(SO₃H);
(xi) DeoxyHex₁Hex₂HexNAc₂NeuAc(SO₃H);
(xii) Hex₂HexNAc₂NeuAc₂;
(xiii) DeoxyHex₁Hex₂HexNAc₂NeuAc;
(xiv) Hex₂HexNAc₂NeuAc₂(SO₃H),
or a part thereof.

38. The method of claim 26 wherein the cancer marker is detected by mass spectrometry.

39. The method of claim 26 wherein the cancer marker is detected by a process comprising contacting an affinity ligand that binds to the cancer marker with the sample for a time and under conditions sufficient for binding to occur and then detecting the binding.

40. The method of claim 39 wherein the affinity ligand is an antibody.

41. The method of claim 39 wherein affinity ligand is a lectin or selectin.
